(19) United States
(12) Patent Application Publication
(10) Pub. No.: US 2023/0005594 A1
(43) Pub. Date: Jan. 5, 2023

(54) CLINICAL DECISION SUPPORT FOR PERSONALIZED ADAPTIVE PROSTATE CANCER THERAPY

(71) Applicant: H. LEE MOFFITT CANCER CENTER AND RESEARCH INSTITUTE, INC., Tampa, FL (US)

(72) Inventors: Heiko ENDERLING, New Tampa, FL (US); Rence BRADY-NICHOLLS, Riverview, FL (US)

(21) Appl. No.: 17/782,897

(22) PCT Filed: Dec. 7, 2020
(86) PCT No.: PCT/US2020/063534

§ 371 Date: Jun. 6, 2022

Related U.S. Application Data
(60) Provisional application No. 62/944,804, filed on Dec. 6, 2019.

(51) Int. Cl. G16H 20/40; G16B 5/30; A61K 31/337; A61K 31/58

(52) U.S. Cl. CPC G16H 20/40; G16B 5/30; A61K 31/337; A61K 31/58

(57) ABSTRACT

Disclosed are methods for personalized treatment of tumor lesions in subject following primary tumor treatment. Disclosed are methods related to using patient specific prostate cancer dynamic model to adjust a patient's intermittent androgen deprivation therapy to determine when to pause IADT, when to resume IADT, the amount of treatment, and the rate of treatment and thereby increasing the time to progression of the tumor and thus prolonging post cancer treatment survival.

This application claims the benefit of U.S. Provisional Application No. 62/944,804, filed on Dec. 6, 2019 which is incorporated herein by reference in its entirety.

This invention was made with government support under Grant No. CA234787 and CA143970 awarded by National Institutes of Health. The government has certain rights in the invention.

### BACKGROUND

Prostate cancer (PCa) is the most prevalent cancer in men in the US. With more than 160,000 men diagnosed with PCa in 2017, of which 30,000 succumbed to their evolving treatment-resistant disease. Following surgery or radiation, the standard treatment for biochemically recurrent PCa is continuous androgen deprivation therapy (ADT) at the maximum tolerable dose (MTD) with or without concurrent docetaxel until the tumor becomes castration resistant. Second line treatment usually includes continuous MTD abiraterone acetate (AA) therapy, although AA was recently approved to be included in first line therapy. Importantly, advanced PCa is not curable because PCa cells are capable of evolving resistance to any current therapy. Thus, here is investigated the molecular and evolutionary dynamics governing resistance and identify strategies that can use this understanding to maximally prolong tumor control.

Prior theoretical, pre-clinical, and clinical studies have questioned the utilization of MTD treatment in non-curative settings. That is, continuous MTD treatment fails to consider the evolutionary dynamics of treatment presponse, where competition, adaptation and selection between treatment sensitive and resistant cells contribute to therapy failure. In fact, this strategy, by maximally selecting for resistant phenotypes and eliminating other competing populations, may actually accelerate the emergence of resistant populations—a well-studied evolutionary phenomenon termed “competitive release.” In part to address this issue, prior trials have used an intermittent on- and off-ADT (IADT) to reduce toxicity and delay time to progression (TTP). However, these trials were typically not designed with a detailed understanding of the underlying evolutionary dynamics. For example, a prospective Phase II trial of IADT for advanced PCa included an 8 months induction period in which the patients were treated at MTD prior to beginning intermittent therapy. Modeling studies found that only a small number of ADT-sensitive cells would typically remain after the induction period significantly reducing the efficacy of subsequent intermittent treatment. Nevertheless, the study demonstrated non-inferiority to continuous ADT despite significantly fewer days on treatment, and 38.5% of patients were still responding after 6 years. Even more striking responses are being observed in the ongoing clinical trial of second-line intermittent AA therapy for castration-resistant PCa (median TTP not reached; at least 33 vs. 13 months at continuous MTD AA, p<0.001). Although highly promising, successful implementation of intermittent PCa therapy requires (i) identifying resistance mechanisms, (ii) predicting responses and (iii) determining potentially highly patient-specific, clinically actionable triggers for pausing and resuming IADT cycles.

### SUMMARY

Disclosed are methods related to using patient specific prostate cancer dynamic model to adjust a patient's intermittent androgen deprivation therapy to determine when to pause IADT, when to resume IADT, the amount of treatment, and the rate of treatment and thereby increasing the time to progression of the tumor and thus prolonging post cancer treatment survival.

In one aspect, disclosed herein are methods of personalized treatment/inhibition/prevention/reduction of prostate cancer recurrence in a subject following primary treatment comprising: a) measuring prostate specific antigen (PSA) levels (such as, for example blood PSA levels) in the patient; b) applying the measured PSA levels to a prostate cancer dynamic model; c) calculating a patient-specific responder parameters; wherein intermittent on-an off-androgen deprivation therapy (IADT) is paused when PSA levels are below patient-specific pre-treatment levels (for example less than 90, 85, 80, 75, 70, 65, 60, 55, 50% of patient specific pretreatment levels); and wherein IADT is resumed when PSA levels are above patient-specific pre-treatment levels (for example, greater than 5, 10, 15, 20, 25, 30, 35, 40, 45, 50, 55, 60, 65, 70, 75, 80, 85, 90, 95, 100% more than patient specific pre-treatment levels). In some aspects, disclosed herein are methods of personalized treatment/inhibition/prevention/reduction of prostate cancer recurrence of any preceding aspect, further comprising measuring androgen-independent prostate cancer stem cell (PcaSC) levels and androgen dependent non-stem prostate cancer cell (PcaC) levels and applying the measured values to step b before proceeding to step c.

In one aspect, the disclosed herein are methods of personalized treatment/inhibition/prevention/reduction of prostate cancer recurrence of any preceding aspect wherein steps a-c are repeated every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60 days, 9, 10, 11, 12, 13, 14, 15, 16, 17, 18, 19, 20, 21, 22, 23, 24, 25, 26, 27, 28, 29, 30 weeks, 6, 7, 8, 9, 10, 11, 12, 18, 24 months.

It is understood and herein contemplated that the disclosed methods can be performed after primary treatment has been completed. It is further understood and herein contemplated that primary treatment of prostate cancer can comprise any treatment regimen determined to be appropriate by the caring physician, including, but not limited to surgical resection of the tumor, neoadjuvant or adjuvant androgen-deprivation therapy, radiation therapy, chemotherapy, and or hormone therapy.

As disclosed herein the disclosed methods allow the physician to adjust the treatment regimen of the patient based on current PSA levels in the patient and allows for continuous adjustment of the dosage and rate of administration of drugs, the length of IADT, the pausing of IADT, and the resumption of IADT. Thus, it is understood and herein contemplated that disclosed herein are methods of personalized treatment/inhibition/prevention/reduction of prostate cancer recurrence of any preceding aspect, wherein the method is performed after the patient begins ADT. In some aspects, ADT can comprise the concurrent administration of docetaxel or abiraterone acetate (AA).

By applying the models disclosed herein, the rate and amount of AA and/or docetaxel can be determined. Thus, in one aspect, disclosed herein are methods of personalized treatment/inhibition/prevention/reduction of prostate cancer recurrence of any preceding aspect, further comprising calculating docetaxel or AA cytotoxicity and adjusting dosage and timing of docetaxel or AA and ADT.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1A shows a model of PCa dynamics during IADT.

FIG. 1B shows patient 4 with continuous response to IADT. Best fit achieved with constant PCaSC fraction (ps=0).

FIG. 1C shows patient 36 with evolving resistance (Ω) to IADT. PSA levels increase during IADT ‘on’ cycle. Best fit achieved with increasing PCaSC fraction (ps>0).

FIG. 1D shows evaluation of the distribution of model parameter of preliminary model fit to 10 individual patient data (5 responders, 5 resistance). PCaSC self-renewal rate ps is significantly lower for treatment responders (resp.) compared to patients developing resistance (resist.) (average 8.7e-06 vs. 0.1; median 0.004 vs. 0.17; p<0.04). No significant difference in other model parameter distributions are observed to fit patient's data.

FIGS. 2A and 2B show a model prediction of resistance to IADT in subsequent treatment cycles. Parameter distributions from model training are used to predict response in subsequent IADT cycles. Black curves show model simulation fits in early cycles. From 100 independent forward-prediction simulations (trajectories; green predict response, red predict resistance) we derive probability of resistance, p(Ω). FIG. 2A shows patient (Pt) 1 with continued response to IADT. Top: Model fit to 1^{st} IADT cycle alone (PSA decrease on ADT and PSA increase in off phase). Correct prediction of continued response in 2^{nd} cycle (p(Ω)=0%) Bottom: Model fit to first 2 IADT cycles; correct prediction of continued response in 3^{rd} cycle. FIG. 2B shows patient (Pt) 101 who developed resistance to IADT in 3^{rd} treatment cycle. Top: Model fit to 1^{st} IADT cycle alone predicts resistance in 2^{nd} IADT cycle in 35/100 simulations (p(Ω)=35%). Bottom: Model fit to 2 cycles; correct resistance prediction in 3^{rd} cycle (p(Ω)=100%).

FIG. 3 shows model simulations of alternative IADT protocols for Patient 36 who became resistant in the 4^{th} cycle of IADT (FIG. 1C). PSA monitored every 4 weeks. Light gray: Model fit to trial data (4 IADT cycles, TTP: 1664 days; on treatment: 1117 days [67%]). Blue: simulation-predicted IADT paused when PSA drops below 50% of pre-ADT level and continues when PSA reaches pre-cycle value (11 cycles, 1820 days, 860 days [47%]). Red: simulation-predicted IADT paused if PSA drops below 90% of pre-ADT level (24 cycles, 1988 days, 870 days [44%]). Dark grey: continuous ADT (756 days).

FIGS. 4A, 4B, 4C, and 4D show parameter distributions and model fits for training patients. (FIGS. 4A and 4B show that the model fits to PSA data and corresponding PCaSC dynamics for (4A) a continuous responder and (4B) a patient who developed resistance during his fourth cycle of treatment. PCaSC population is rapidly increasing in resistant patient and slowly in responsive patient due a significantly higher self-renewal rate (pₛ=0.0052 and 0.1349 for patients 029 and 101, respectively) FIG. 4C shows simulated vs. measured PSA. Linear regression obtains an R2 of 0.74. FIG. 4D shows parameter distributions, with φ and ρ uniform between all training patients. Stem cell self-renewal p′ and ADT cytotoxicity α exhibit exponential relationship.

FIGS. 5A, 5B, 5C, 5D, 5E, and 5F show model validation on testing patients. (FIGS. 5A and 5B show that the model fits to PSA data and corresponding PCaSC dynamics for (5A) a continuous responder and (5B) a patient who developed resistance during his third cycle of treatment. PCaSC population is rapidly increasing in resistant patient and slowly in responsive patient due a significantly higher self-renewal rate (pₛ=0.0201 and 0.1118 for patients 091 and 033, respectively). FIG. 5C shows simulated vs. measured PSA. Linear regression obtains an R₂ of 0.69. FIG. 5D shows parameter distributions for the stem cell self-renewal p₂ and ADT cytotoxicity α, with φ and ρ learned from training patients show similar trend found in training cohort. FIG. 5E shows that the model predicted responsiveness in cycles two (98% of simulations) and three (90.3% of simulations) for Patient 017. The probability of resistant (P(Ω)) was less than its respective k (k_{N}=0.45, k_{O}=0.29) for each cycle. He completed the trial on day=2202. FIG. 5F shows that the model predicted resistance in 23.5% of cycle 2 simulations and in 46.6% of cycle 3 simulations for Patient 054. The predicted probability of resistance in cycle three was greater than κ_{O} so he would be advised to stop the trial. Data showed that he became resistant on day=1384 during the third cycle, as predicted.

FIGS. 6A, 6B, and 6C show that Administering IADT without induction period increases TTP when compared to IADT with induction and continuous ADT. FIG. 6A shows Kaplan-Meier estimates of progression comparing the Bruchovsky IADT protocol with induction (black curve) against simulated IADT without induction (blue) and continuous ADT (gray) (^{§} indicates predictive model simulation). With and without induction period, IADT TTP significantly increases when compared to continuous therapy TTP. FIG. 6B shows TTP comparison between Bruchovsky IADT with induction (black), continuous therapy (gray), and IADT without induction (blue). Bruchovsky patients who were lost to follow up are shown in red. Open circles denote end of simulation (EOS). Solid lines denote mean TTP (41.18, 42.83, and 46.9 months for Bruchovsky IADT with induction, continuous therapy, and Bruchovsky without induction, respectively). Though median TTP was not reached for either IADT protocol (6A), the average TTP is longer with IADT without induction, compared to IADT with induction and continuous ADT. FIG. 6C shows the model fit to Patient 054 (Bruchovsky IADT protocol with induction, black curve), simulation without induction (blue curve), and simulation of continuous ADT (gray). On the Bruchovsky protocol, the patient became resistant after 1384 days. Simulating continuous ADT would result in progression after 728 days. Simulating IADT without the induction would increase TTP by 7 months. PCaSC dynamics show that treatment selects for PCaSC population, accelerating resistance development. Dashed line represent time when ADT is off.

FIGS. 7A, 7B, and 7C show that alternative threshold therapy can significantly improve TTP. FIG. 7A shows Kaplan-Meier estimates of progression comparing the Bruchovsky IADT protocol (black curve) with alternative therapy protocol using a threshold of 10% (maroon), 50% (green), and 70% (blue) (§ indicates predictive model simulation). A 50% or 70% threshold can significantly increase TTP when compared to the Bruchovsky IADT protocol. FIG. 7B show TTP comparison between Bruchovsky IADT (black) and thresholds of 10%, 50%, and 70%. Bruchovsky patients who were lost to follow up are shown in red. Open circles denote end of trial/simulation (EOS). Solid lines denote mean TTP (41.18 and 41.92 for Bruchovsky IADT and 10% trigger, respectively). FIG. 7C shows alternative threshold therapy simulations for Patient 054. On the trial protocol, the patient became resistant after 1384 days. With a threshold of 10%, resistance could be delayed for 1554 days. With a threshold of 50% and 70%, the patient could continue on the protocol for more than 3600 days.

FIGS. 8A, 8B, 8C, 8D, and 8E show Docetaxel administration applied prior to or after IADT progression can increase TTP. FIG. 8A shows that in those patients given docetaxel prior to IADT progression, patients with high p₂ receive the highest benefit. FIG. 8B shows Kaplan-Meier estimates of progression comparing Bruchovsky IADT protocol (black) against castration naïve (maroon) and castration resistant (blue) docetaxel simulations (§ indicates predictive model simulation). Castration naïve and resistant refer to docetaxel given prior to IADT and after IADT progression, respectively. Six cycles of docetaxel prior to IADT increased TTP by 21.8 months on average. Ten cycles of docetaxel after IADT progression increased TTP by 17.5 months on average. FIG. 8C shows TTP comparison between Bruchovsky IADT (black) and castration naïve (maroon) and castration resistant (blue) docetaxel simulations. Bruchovsky patients who were lost to follow up are shown in red. Open circles denote end of trial/simulation (EOS). Administering docetaxel after IADT progression significantly increases TTP. Solid lines denote mean TTP (41.18, 43.92, and 50.14 months for Bruchovsky IADT, castration naïve, and castration resistant, respectively). Comparison of TTP shows that late DOC administration can significantly extend TTP when compared to IADT alone. FIGS. 8D and 8E show castration naïve (red) and castration resistant (blue) simulations. FIG. 8D shows that on the Bruchovsky IADT protocol, Patient 012 developed resistance after 839 days. Administering docetaxel prior to IADT delayed progression for an additional 27 months, while docetaxel given after progression allowed the patient to remain on treatment for an additional 16 months. FIG. 8E shows that though the trial concluded after 6.5 years, simulations shows that Patient 024 could have remained on the Bruchovsky IADT protocol for at least 10 years. Giving docetaxel prior to IADT produced similar results.

FIGS. 9A, 9B, 9C, 9D, and 9E show first cycle ps stratifies patients who could benefit from docetaxel after first cycle of IADT. FIG. 9A shows p2 distributions between patients who may benefit from docetaxel after first cycle of IADT and those who may not. Median p2 stratifies patients who could benefit from docetaxel after first cycle of IADT. FIG. 9B shows Kaplan-Meier estimates of progression with and without docetaxel after first cycle of IADT (§ indicates predictive model simulation). Stratifying by median p2 shows that patients with p2≥med(p2) (red) experience greatest benefit from docetaxel (gain of 6 months). For patients with with p2<med(p2) (green), there was not a significant benefit in TTP. FIG. 9C shows TTP comparison between Bruchovsky IADT with and without docetaxel. Open circles denote end of simulation (EOS). TTP was significantly lower in patients with p2≥med(p2) both with and without docetaxel. Solid lines denote median TTP (62.98, 62.27, 34.13, and 38.15 months (left to right) for patients with low p2 (green) and high p2 (red), respectively). FIGS. 9D and 9E show simulation results of Bruchovsky IADT with (gray) and without (black) docetaxel after first cycle. FIG. 9D shows Patient 084 (p2=0.0386>med(p2)) gains at least 18 months with docetaxel after first cycle. FIG. 9e shows Patient 016 (p2=0.0017<med(p2)) did not progress with or without docetaxel.

FIGS. 10A, 10B, 10C, and 10D show cycle to cycle parameter changes. FIG. 10A shows cycle to cycle parameter distributions for p′ and a (changes between cycles is not significant (p>0.05). FIG. 10B shows cumulative probability of relative changes in p′ between cycles. FIG. 10C shows α vs. p′, fit (black curve), and 95% confidence interval (gray). FIG. 10D shows receiver operating curves for training predictions for cycles two through four. The resistance threshold, K, that maximizes the accuracy and hence, predictive power, is shown in red.

FIG. 11 shows treatment times for patients included in the study. Sixteen patients were included in the model analysis. Gray and white denote when treatment was on and off, respectively. Red triangles and black x's denote when patient developed PSA and radiographic progression, respectively.

FIGS. 12A, 12B, 12C, and 12D show leave-one-out analysis results. Model fits to PSA data and corresponding stem cell dynamics for a (12A) continuous responder and (12B) a patient who developed resistance in his third cycle of treatment. Parameter distributions for (12C) patient-specific parameters pₛ and α and (12D) uniform model parameters viand p determined via leave-one-out analysis.

FIGS. 13A, 13B, and 13C show model prediction results. FIG. 13A shows prediction classifier table. Model predictions for (13B) Patient 1011 a continuous responder and (13C) Patient 1014 who progressed in the 3^{rd} cycle. The model correctly predicted that Patient 1011 would continue to respond in both the 2^{nd} and 3^{rd} cycles and that Patient 1014 would continue to respond in the 2^{nd} cycle and develop resistance in the 3^{rd} cycle.

FIGS. 14A and 14B show model prediction results when considering metastatic burden. FIG. 14A shows parameter sampling with and without metastases. When a patient's metastatic burden is not considered, a uniform sampling is performed to determine pₛ value in next cycle. If a patient develops a new metastatic growth, the sampling is skewed such that the sampled stem cell self-renewal value is more likely to be larger in the next cycle. FIG. 14B shows model predictions for Patient 1010 with and without incorporating metastatic burden. Patient 1010 developed one new metastatic growth during his first off-treatment cycle. Using PSA dynamics alone, the model incorrectly predicts that Patient 1010 will continue to respond. Using a skewed sampling results in a correct prediction that Patient 1010 will progress in cycle 2.

FIG. 15 shows Model of PCaSC and non-stem (differentiated) cell dynamics Interactions between PCaSCs and differentiated cells, as well as serum PSA. PCaSCs can divide asymmetrically to produce differentiated cells. PCaSCs divide at rate λ, self-renew at rate pₛ. The differentiated cells inhibit the production of PCaSCs, die in response to ADT at rate α (shown by the red arrow), and produce PSA at rate p. PSA naturally decays at rate ψ. Unlike ADT, chemotherapy causes cell death in both the PCaSCs and differentiated cells (yellow arrows) at rates δₛ and δ_{D}, respectively.

FIGS. 16A and 16B show model calibration results. Model fits to longitudinal PSA data for a (16A) castration naïve and (16B) castration resistant patient. Patient 109 received six cycles of chemotherapy concurrently with ADT followed by ADT alone, while Patient 105 received ADT alone, then eight cycles of concurrent therapy, followed by ADT alone. The bottom panels show that the PCaSCs decline during concurrent therapy. The model is able to describe each patient's data equally well.

FIGS. 17A and 17B show model simulations of early vs late concurrent treatment. Model simulations using the optimal values for each individual patient. FIG. 17A shows patient 109 was castration naïve and received chemotherapy with ADT at the start of treatment. Model simulations show that he could benefit from later chemotherapy administration (black curve). FIG. 17B shows patient 105 was castration resistant, that is he received concurrent therapy after developing resistance to ADT alone. Model simulations show that early concurrent therapy would result in earlier progression (gray curve).

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Controlled release” or “sustained release” refers to release of an agent from a given dosage form in a controlled fashion in order to achieve the desired pharmacokinetic profile in vivo. An aspect of “controlled release” agent delivery is the ability to manipulate the formulation and/or dosage form in order to establish the desired kinetics of agent release.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Methods of Personalized Prostate Treatment

Prostate cancer (PCa) is the most prevalent cancer in men in the US. About 1 in 9 American men will be diagnosed with PCa in his lifetime (>190,000 estimated in 2020) and 1 in 41 will die from it. Consequently, PCa remains the second leading cause of death in American men. Following surgery or radiation treatment, androgen deprivation therapy (ADT) has been the mainstay treatment for hormone sensitive PCa for over 70 years. ADT suppresses the production of testicular androgen, which both the normal and cancerous cells depend on for survival and proliferation. Despite new strategies in “precision medicine” in which the specific therapy is guided by molecular biomarkers, treatment protocols are typically based on the conventional strategy of “maximum tolerated dose until progression”. This often results in the competitive release of the resistant phenotype, leading to early treatment progression. In an effort to delay progression, intermittent androgen deprivation treatment (IADT) has been shown to be a promising alternative that reduces toxicity and delays progression in many patients. Despite this advance, patients inevitably develop castration resistant prostate cancer, which often progresses to metastatic castration resistant prostate cancer (mCRPC).

Second-line hormone therapy abiraterone acetate, which inhibits the production of androgens from other cells by blocking the protein CYP17, has been shown to prolong overall survival when compared to prednisone or placebo. The PSA response rate to abiraterone plus gonadotropin-releasing hormone analog was 62% with a median time to progression of 11.1 months in the AA-302 trial. It has been proposed that adaptively administering abiraterone in mCRPC, allowing for treatment holidays when a patient has sufficiently responded, may be able to increase overall response and delay progression. However, maximizing the benefit of adaptive therapy requires understanding the dominant drivers of resistance, predicting individual patient responses, and identifying when and how to modulate treatment to maximize response time.

We have developed a quantitative model of prostate cancer stem cell (PCaSC), non-stem cells, and prostate-specific antigen (PSA) dynamics to describe the response to IADT in biochemically recurrent PCa patients. Model analysis identified stem cell enrichment as a plausible driver of resistance. With this finding, we used early treatment evolutionary dynamics to predict how patients would respond to subsequent cycles of treatment with an overall accuracy of 89%. For this study, we sought to extend the model to the mCRPC setting and determine whether or not this simple model can describe and predict treatment dynamics. To do so, we calibrated and validated the model against longitudinal PSA data from 16 mCRPC patients receiving adaptive abiraterone in a trial (NCT02415621). We then used early treatment dynamics to predict how individual patient respond to treatment. As these patients are metastatic, we also investigated how metastatic burden can be utilized to maximally predict patient response.

1. Enrichment in Androgen-Independent PCa Stem Cells (PCaSC) Contribute to IADT Resistance, and Simulating PCaSC Dynamics in Early IADT Treatment Cycles to Computationally Forecast Patient-Specific Disease Dynamics and Prostate Specific Antigen (PSA) Serum Levels Reliably Predict IADT Response or Resistance in Subsequent Treatment Cycles.

To solve this complex problem an innovative framework can be used to simulate and predict the dynamics of androgen-independent PCaSC, androgen-dependent non-stem PCa cells (PCaC), and blood PSA concentration during IADT. Mathematical model simulations of PCaSC enrichment can be fitted to retrospective longitudinal PSA measurements in individual patients to identify disease dynamics of patients whose tumors became treatment resistant. These analyses identify optimal individual patient PSA cutoff values to trigger treatment on/off decisions to maximally delay onset of resistance and TTP. With this dynamic framework PCaSC and PSA dynamics can be determined as putative triggers to personalize IADT and maximize TTP, ultimately to improve PCa outcomes. While this developmental project focuses on PCa, the concept and methodologies are translatable to multiple other cancers that become resistant to continuous MTD therapy.

a) Establish an Innovative in Silico Framework to Simulate PCaSC Dynamics and Predict PCa Response to IADT.

53. An in silico model of PCaSC enrichment and PSA dynamics during IADT can be developed. Model parameters can be derived from a retrospective training set of longitudinal PSA data of patients who either remained responsive (N=17) or became resistant (N=10) to IADT. Model calibrations can be assessed in a validation data set (17 responsive, 11 resistant). Model ability to predict evolution of PCa dynamics and IADT response can be scored and PCaSC and PSA dynamics confirmed as actionable biomarkers of response.

b) Simulate Patient-Specific PSA Cutoff Values as on/Off Triggers for IADT Cycles to Maximally Delay TTP.

The validated model(s) can be simulated for each patient with different IADT cycle intervals. Fixed cutoffs for PSA values (4-10 ng/mL) as well as relative PSA levels (0-100% of pre-treatment PSA value) to pause and resume IADT can be simulated to determine predicted TTP. PSA cutoffs that maximize TTP can be correlated with PCaSC dynamics parameters and used to identify optimal patient-specific IADT protocols.

c) Assess Optimal Timing of Concurrent Docetaxel with IADT.

Docetaxel (DOC) combined with ADT improves overall survival and TTP where benefit depends on treatment timing. DOC therapy initiated at different IADT cycles (first, second, etc.) can be simulated, and determinations of DOC timing-dependent TTP in correlation to patient-specific PCaSC dynamics in early IADT cycles.

2. Significance.

Prostate cancer is the most prevalent cancer in men in the US, and continuous ADT at MTD has been the standard of care for many decades. Despite this, as noted by the NIH (NIH cancer.gov website), “doctors cannot predict how long hormone therapy can be effective in suppressing the growth of any individual man's prostate cancer.” The ability to forecast PCa treatment response and the onset of resistance is the key to dynamically adapting therapy before resistance emerges. IADT with on- and off-treatment cycles is an alternative to continuous ADT at MTD. IADT has two major advantages. It (1) reduces drug use by more than 50%, reducing deleterious side effects, including psychological trauma, hot flashes, sexual dysfunction, loss of libido, and reductions in bone density; and (2) introduces treatment holidays that counteract evolutionary dynamics by allowing treatment-sensitive populations to relapse and successfully compete with growing treatment-resistant clones. In prospective clinical trials, IADT was shown to be no poorer than continuous ADT; but superiority has yet to be confirmed. This is due, at least in part, to the long “induction periods” in which continuous ADT at MTD is administered for 7-8 months thus preventing evolutionary competition between treatment-sensitive and treatment-resistant populations during intermittent treatments. The second-line intermittent AA therapy trial in which treatment is cyclical but based on mathematical models of the underlying evolutionary dynamics (cycle length ranging from 4 to 14 months) showed significant improvements in TTP (median not reached; at least 33 vs. 14 months [in a comparable contemporaneous cohort treated with continuous MTD, ADT] p<0.001).

Here this work is extended through the explicit integration of cancer stem cell (CSC) biology in the dynamics of therapy response and resistance. CSCs have been identified in a number of human tumors including PCa. PCaSC are long-lived, treatment resistant, can initiate and propagate tumors, and give rise to heterogeneous tumor populations after treatment. An increase in CSC self-renewal rates is a mechanism of CSC enrichment during prolonged cancer radiotherapy. Similar mechanisms may be at play during continuous PCa therapy, and treatment-induced selection for androgen-independent PCaSC may be causal for therapy resistance, as proposed at the 2017 AACR Prostate Cancer conference. PCaSC dynamics can be investigated as resistance mechanisms and integrate these into quantitative models that predict and optimize responses to IADT for individual patients with a specific goal of identifying clinically actionable cues for pausing and resuming IADT cycles. These platforms significantly improve PCa outcomes through personalizing treatment that maximally delays TTP while lowering cumulative dose and thereby limiting treatment side effects.

3. Innovation

Even highly effective cancer therapy almost invariably fails due to evolution of resistance. Current cancer treatment with continuous application of drugs at MTD actually accelerates evolution of resistance. Furthermore, despite new strategies in “precision medicine” in which the specific therapy is guided by molecular biomarkers, treatment protocols rarely vary between patients. To date there is no attempt to identify and counteract the patient-specific dynamics of intratumoral evolution of resistance during therapy. For example, despite the advance of IADT to reduce PCa treatment dose and delay TTP, therapy is currently paused and resumed based on fixed PSA values that are determined before therapy and are not patient specific. Cancer is a complex, dynamic system best understood by analyzing the response to external perturbation and selection forces—such as therapy. Shown herein is a novel and innovative framework to identify PCaSC dynamics underlying the evolution of PCa resistance and rising blood serum PSA levels, and use PCa dynamics and treatment response patterns as clinically actionable cues to inform treatment cycles.

Because of its complexity, this approach must be multidisciplinary. This research program is the first attempt to calibrate and validate a mathematical model of PCaSC dynamics during therapy to forecast patient-specific responses to subsequent treatment cycles, and to use this to identify optimal longitudinally collected PSA values to aid treatment decisions. After the successful trial of intermittent AA for castration-resistant PCa (NCT02415621), the next logical steps for clinical trials include the investigation of different PSA values (patient-specific; 80% of pre-treatment PSA value; IRB pending) to further delay TTP. These clinical studies provide additional, independent data for validating the project. This study's framework provides the rationale, proof-of-principle and required sample size for both non-inferiority and superiority of subsequent first-in-kind clinical trials of patient-specific treatment triggers based on dynamic properties derived from a mathematical model (for which additional funding can be sought). This holds key to significantly improve cancer outcomes, first in PCa and then in other cancers currently incurable with continuous MTD therapy.

4. Specific Approach.

Provided herein is an innovative Quantitative Personalized Oncology approach, aimed at integrating patient-specific longitudinal PSA data (collected every 4 weeks until treatment resistance) to iteratively develop, calibrate, and validate a mathematical model of PCa response dynamics during IADT. The calibrated and validated model can be fit to an individual patient's data to derive model parameters required to predict patient-specific responses. In silico trials of alternative clinical decision triggers based on individual response dynamics informing individual on/off IADT cutoffs can be simulated. IADT triggers are identified herein to optimize response and maximally delay TTP on a per patient basis. The model system can be used to explore benefits of concurrent docetaxel chemotherapy and identify optimal sequencing and timing of IADT and docetaxel.

In one aspect, disclosed herein are methods of personalized treatment/inhibition/prevention/reduction of prostate cancer recurrence in a subject following primary treatment comprising: a) measuring prostate specific antigen (PSA) levels (such as, for example blood PSA levels) in the patient; b) applying the measured PSA levels to a prostate cancer dynamic model; c) calculating a patient-specific responder parameters; wherein intermittent on-an off-androgen deprivation therapy (IADT) is paused when PSA levels are below patient-specific pre-treatment levels (for example less than 90, 85, 80, 75, 70, 65, 60, 55, 50% of patient specific pretreatment levels); and wherein IADT is resumed when PSA levels are above patient-specific pre-treatment levels (for example, greater than 5, 10, 15, 20, 25, 30, 35, 40, 45, 50, 55, 60, 65, 70, 75, 80, 85, 90, 95, 100% more than patient specific pre-treatment levels). In some aspects, disclosed herein are methods of personalized treatment/inhibition/prevention/reduction of prostate cancer recurrence of any preceding aspect, further comprising measuring androgen-independent prostate cancer stem cell (PcaSC) levels and androgen dependent non-stem prostate cancer cell (PcaC) levels and applying the measured values to step b before proceeding to step c.

In one aspect, the disclosed herein are methods of personalized treatment/inhibition/prevention/reduction of prostate cancer recurrence of any preceding aspect wherein steps a-c are repeated every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60 days, 9, 10, 11, 12, 13, 14, 15, 16, 17, 18, 19, 20, 21, 22, 23, 24, 25, 26, 27, 28, 29, 30 weeks, 6, 7, 8, 9, 10, 11, 12, 18, 24 months.

It is understood and herein contemplated that the disclosed methods can be performed after primary treatment has been completed. It is further understood and herein contemplated that primary treatment of prostate cancer can comprise any treatment regimen determined to be appropriate by the caring physician, including, but not limited to surgical resection of the tumor, neoadjuvant or adjuvant androgen-deprivation therapy, radiation therapy, chemotherapy, and or hormone therapy.

As disclosed herein the disclosed methods allow the physician to adjust the treatment regimen of the patient based on current PSA levels in the patient and allows for continuous adjustment of the dosage and rate of administration of drugs, the length of IADT, the pausing of IADT, and the resumption of IADT. Thus, it is understood and herein contemplated that disclosed herein are methods of personalized treatment/inhibition/prevention/reduction of prostate cancer recurrence of any preceding aspect, wherein the method is performed after the patient begins ADT. In some aspects, ADT can comprise the concurrent administration of docetaxel or abiraterone acetate (AA).

By applying the models disclosed herein, the rate and amount of AA and/or docetaxel can be determined. Thus, in one aspect, disclosed herein are methods of personalized treatment/inhibition/prevention/reduction of prostate cancer recurrence of any preceding aspect, further comprising calculating docetaxel or AA cytotoxicity and adjusting dosage and timing of docetaxel or AA and ADT.

5. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A.R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone can range from about 1 ug/kg to up to 100 mg/kg of body weight or more per day, depending on the factors mentioned above.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

a) Establish a Mathematical Model of PCaSC Dynamics and Predict PCa Response to IADT.

109 patients with localized and biochemically recurrent PCa were treated as part of the Canadian prospective Phase II trial of IADT with 6 years of follow-up. PSA was monitored every 4 weeks, and IADT was paused if PSA<4 ng/mL for consecutive 36 weeks. Patients resumed IADT if PSA>10 ng/mL. Clinical trial investigators have shared the de-identified data (patient-specific longitudinal PSA and testosterone values, treatment on/off, clinical notes for treatment failure) of N=55 patients who each underwent at least 2-4 cycles of IADT (34 patients finished the trial with continued response [responders], 21 patients evolved resistance [SI, resistant]).

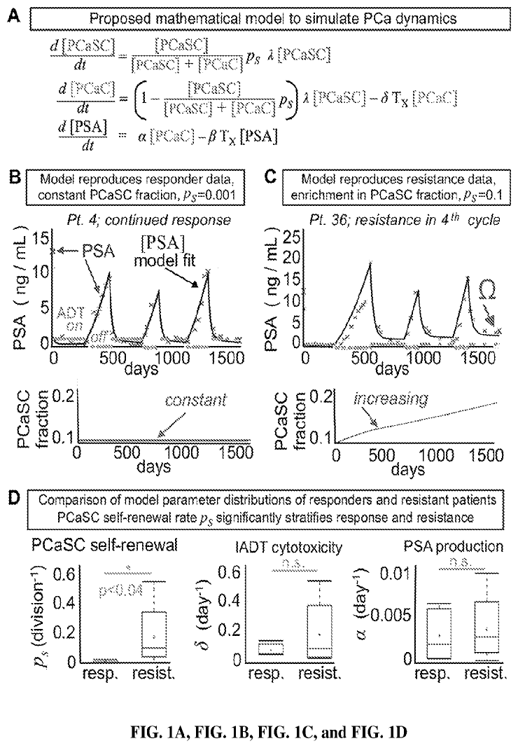

A mathematical model of PCa dynamics and blood serum PSA levels during IADT was developed (FIG. 1A). Tumors are heterogeneous populations of PCaSC and non-stem, more differentiated PCaC. PCaSC proliferate with an intrinsic rate, λ day. Symmetric PCaSC division and self-renewal occurs with frequency pₛ with negative feedback from the PCaC cell population (pₛ*[PCaSC]/([PCaSC]+PCaC])).

83. PCaC are produced during asymmetric PCaSC division events, and eliminated with rate δ day⁻ during on-treatment cycles. PSA is produced with rate α day⁻ by PCaC, and depleted with rate β day⁻ during treatment. IADT on-treatment cycles are simulated with T_{X}=1; off treatment is simulated with T_{X}=0. Model simulations of the PCa dynamics and resulting PSA levels fit to the longitudinal data of 10 patients (5 responders, 5 resistant) with high accuracy (R²=0.84; 4 model parameters; 17-22 data points per patient). Patients that developed resistance (Ω) showed significant increase in the PCaSC fraction during IADT (FIGS. 1B and 1C). Analysis indicates that of the five model parameters only PCaSC self-renewal rates (pₛ) are significantly higher in resistant patients compared to continued responders (FIG. 1D). These data strongly support a model of PCaSC dynamics early during IADT can be used to simulate subsequent patient-specific treatment responses.

(1) Specific Approach:

PCaSC self-renewal dynamics during IADT treatment cycles fits the data of patients that continue to respond (median pₛ=0.004) and patients that develop resistance (median pₛ=0.17). Self-renewal was simulated to be modulated by the negative feedback of PCaC on the PCaSC population, which reduces during IADT cycles and leads to selection of androgen-independent PCaSC.

(2) Data Fitting and Parameter Determination:

Available retrospective clinical data (N=55) can be randomly allocated to model training (17 responders, 10 resistant) and validation (17 responders, 11 resistant). The mathematical model (FIG. 1A) can be solved numerically in Matlab (Mathworks, Natick, Mass.). Model simulated PSA values can be fit to the longitudinal PSA data of patients in the model-training cohort. Briefly, a genetic algorithm is applied to derive the five model parameters (PCaSC proliferation rate λ and self-renewal rate pₛ, PCaC androgen-deprivation sensitivity δ, PSA production rate α and treatment-induced PSA depletion rate β) such that the model-simulated PSA values best fit the longitudinal data of each patient. The fitness (cost) of each stochastically derived parameter set {λ, p_{S}, δ, α, β} is calculated by summing the absolute values of residuals between data and corresponding simulation results. The 1000 parameter sets that minimize the residual sums after 1000 algorithm iterations are refined with a trust-region reflective algorithm implemented in the MATLAB lsqnonlin function. Parameter distributions can be derived and analyzed for resistant patients and responders as described above (FIG. 1D). Differences in parameter distributions between patient cohorts can be assessed using the nonparametric Mann-Whitney U test with p<0.05 deemed significant. Significantly different parameter values between both cohorts can indicate mechanisms that drive resistance.

b) Treatment Prediction and Model Validation.

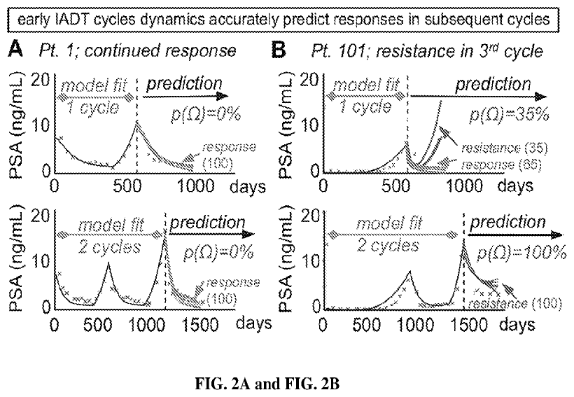

Model-associated parameter distributions can be determined by fitting simulation results to longitudinal PSA data (every 4 weeks for up to 6 years) of individual patients in the training set. For each patient in the validation cohort, Bayesian interference approaches are deployed to predict response. The model is fit to the first treatment cycle (then first two, first three, etc.) to derive patient-specific parameters. The parameter variations observed are applied in the training cohort to the patient-derived parameter set and forecast model dynamics in the subsequent treatment cycle (FIG. 2A). This is repeated 100 times to derive the proportion of simulations that resistance in the subsequent on-treatment cycle. From this the probability of resistance, p(Ω) is derived. Resistance is defined as sequential increases of PSA above normal range (4 ng/mL) during IADT. Data shows a prediction dichotomy for some cycles, p(Ω)=35% (FIG. 2B).

The ability of p(Ω) to discriminate patient response from resistance can be assessed using the area under the ROC curve (AUC) in the validation data. Assuming 17 responsive and 11 resistant patients, 80% power is had to detect an AUC of 0.80 using the method of DeLong et al. An optimal cutoff value for p(Ω) to stratify prediction into response/resistance with highest accuracy can be determined by maximizing Youden's J Index (=sensitivity+specificity−1) in the training data. To assess clinical utility, the positive predictive value (PPV) and negative predictive value (NPV) for the optimal p(Ω) cutoff can be calculated in the validation set.

Technical expertise to develop models of cancer biology and stem cell biology are established, and software (Matlab; Statistics, Optimization, & Machine Learning toolboxes) and algorithms to fit model simulations to patient-specific data and determine model parameters are available in the group and can be adapted for this project. Analysis of each 5 responders and resistant patients led to statistical significance in difference in PCaSC self-renewal rates (FIG. 1). To assess the robustness of estimated prediction performance in the primary analysis, a sensitivity analysis can randomize varying numbers of patients into model training, up to leave-one-out studies. If model fit to data is consistently insufficient (R²<0.75) or the models' predictive power unsatisfactory (Youden's J Index<0.75), different or additional terms can be considered for the mathematical model, including but not limited to explicit consideration of androgen levels (data available but not included in the model for sake of simplicity), androgen concentration-dependent cell turnover rates, androgen-dependent PSA production rate, or consideration of PCaSC as androgen producers or replacement with an androgen producing population.⁵ Alternative treatment-induced enrichment of androgen-independent PCaSC can come from lineage plasticity and de-differentiation of PCaC during androgen deprivation. This lineage plasticity model can be similar to the model (FIG. 1A) but with a constant PCaSC self-renewal rate pₛ≥0 and phenotypic transition of PCaC into the PCaSC compartment (with function ƒ([PCaSC], [PCaC]) that can be a fixed rate or dependent on both PCaSC and PCaC populations).

c) Simulate Patient-Specific PSA Values as on/Off Triggers for IADT Cycles to Maximally Delay Time to Progression (TTP).

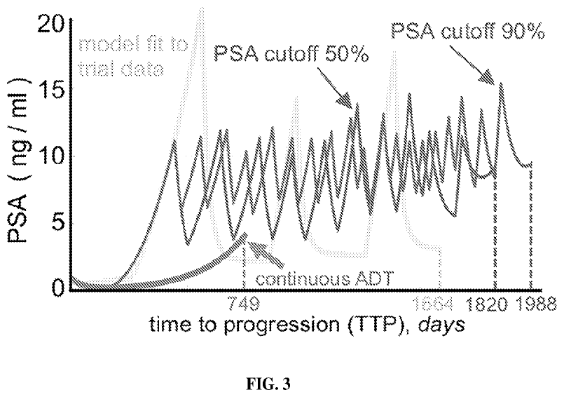

Prospective clinical trials have shown that IADT is not inferior in overall survival despite significantly fewer on-treatment days and lower cumulative doses. Survival benefits have not been confirmed clinically, which can be due to the conservative trial designs of long on-treatment cycles (7-8 months) and short off-cycles. Such long treatment periods would eliminate nearly all sensitive cells, thereby promoting competitive release of more resistant cells. To better counteract evolutionary dynamics, the ongoing trial of intermittent second-line abiraterone therapy stopped on-treatment cycles when PSA fell below 50% of its pre-treatment value until PSA returned to its initial level. This yielded significantly shorter treatment cycles (median 4.5 months) and significant improvement in TTP (median not reached, at least 33 vs. 16.6 months at continuous ADT, p<0.001). These results motivate the hypothesis that shorter on-treatment phases can also improve first-line therapy IADT outcomes with patient-specific PSA values to trigger treatment on/off cycles. Treatment response to different IADT protocols was simulated using the individual parameters for Patient 36 who became resistant in the 4^{th} IADT cycle in the Canadian trial. PSA values can be monitored every four weeks as done in the clinic. The data indicate that TTP is highly dependent on IADT on/off triggers, with continuous ADT yielding the shortest TTP (FIG. 3). For Patient 36, relative PSA values of 50% or 90% of pre-treatment PSA levels as triggers to pause IADT and pre-treatment PSA levels to resume IADT yield longer TTP than the static triggers (PSA monitored every 4 weeks, IADT is paused if PSA<4 ng/mL for consecutive 36 weeks, and IADT resumes if PSA>10 ng/mL). In the trial, TTP for patient 36 was 1664 days with 1117 days (67%) on treatment; predicted TTP is 1820 days with 860 days (47%) on treatment with IADT off at 50% initial PSA; and predicted TTP is 1988 days with 870 days (44%) on treatment with IADT off at 90% initial PSA. These data indicate that different relative PSA values as triggers for on/off therapy yield different IADT cycle lengths and significantly different TTP. An optimal trigger for IADT on/off decisions exists, and can maximally delay response and prolong TTP, which can be dependent on patient-specific PCaSC dynamics.

(1) Specific Approach:

The validated mathematical model can be simulated using different IADT cycle on/off triggers for each patient with their individually derived model dynamics and parameters. Fixed PSA values (0.5-10 ng/mL) as well as relative PSA values (0-100% of pre-treatment PSA value) to initiate and pause IADT can be simulated. For each treatment simulation the number of treatment cycles and TTP (sequential increases of PSA≥4 ng/mL on-therapy) are analyzed. Treatment protocols that transition responding patients under the clinical trial protocol to become resistant under the investigational protocol can be rejected. Simulated treatment protocols that shorten TTP for at least one patient can also be rejected. For each patient, the optimal cutoff for PSA values to trigger IADT on/off cycles can be determined by identifying the strategy that results in maximum simulated TTP. To investigate how to personalize and optimize treatment after initial treatment response assessment (i.e., treat for one cycle with standard triggers and fit the model to this first treatment cycle alone), optimal PSA cutoff values to trigger IADT on/off in subsequent treatment cycles can be derived and correlated with the first treatment cycle response and model parameter for PCaSC dynamics on a per-patient basis. Progression-free survival after static PSA cutoffs from the Canadian trial as well as simulated TTP under the project-derived optimal PSA cutoffs can be visualized via Kaplan-Meier curves.

(2) Bayesian Adaptive Approach:

The increase of the androgen-independent PCaSC contributes to treatment resistance. As the total number and relative proportion of the PCaSC population is changing during on and off IADT cycles, treatment decision triggers can also change. Dynamically adapting PSA values can be anyzed as a function of PCaSC parameter evolution with each treatment cycle and determine optimal treatment triggers to maximize TTP. In brief, following an Bayesian adaptive prediction approach the mathematical model can be applied to the first treatment cycle alone and derive the optimal PSA cutoff to trigger IADT on/off cycles. After each subsequent treatment cycle, the model can be fit o all treatment cycles to this point (2^{nd}, 3^{rd}, etc.) and derive the optimal PSA cutoff to trigger IADT on/off in the next cycle. The model-predicted TTP following this adaptive approach can be compared to the non-adaptive optimal PSA cutoffs uniformly through the treatment. TTP for Bayesian adaptive and non-adaptive PSA cutoffs can be visualized via Kaplan-Meier curves.

As the data demonstrates, the necessary technical expertise is in place to successfully execute the modeling parts of the study. Patient-specific triggers for IADT on/off decision exist, and that these triggers can be correlated with PCaSC dynamics obtained in the initial cycle(s) of IADT. When PCaSC self-renewal dynamics cannot be correlated with treatment response and corresponding triggers, other model mechanisms (PCaC androgen-deprivation sensitivity, rate δ, PSA production rate α and treatment-induced PSA depletion rate β) can be evaluated either alone or in combination with PCaSC dynamics. When the inter-patient variation in optimal treatment triggers is low, the analysis can be repeated for different absolute or relative PSA triggers for optimal therapy simultaneously for all patients to derive the best possible protocol for the entire patient population. Selecting optimal PSA cutoffs on the sole consideration of maximal TTP may not be ideal for all patients, as undesirably long treatment windows may be chosen in some cases.

d) Evaluate the Timing-Dependent Benefit of Concurrent Docetaxel with IADT.

Docetaxel (DOC) is a chemotherapy drug that is administered every three weeks over ten or more cycles. DOC combined with ADT showed improved patient survival with survival benefit dependent on timing. Compared to ADT alone, adding docetaxel at the beginning of long-term PCa hormone therapy increased overall survival time by 10-13 months (81 vs. 71 months), 57.6 vs. 44 months) with evidence of prolonged TTP (20.2 months vs. 11.7 months). Adding docetaxel at the end of ADT when PCa becomes resistant (hormone-refractory PCa) increased survival by only 2.4 months (18.9 vs. 16.5 months). These data demonstrate that docetaxel added to ADT at different times can yield different outcome benefits.

(1) Specific Approach.

Docetaxel promotes and stabilizes microtubule assembly, thereby decreasing free tubulin needed during cell division and leading to cell apoptosis independent of androgen availability. Thus, androgen-independent PCaSC are sensitive to docetaxel, albeit to a lesser degree than non-stem PCa cells. A [DOC] compartment can be introduced into the model, which receives a bolus increase and times when DOC is administered, and [DOC] concentration decays with DOC-associated PK/PD parameters. Docetaxel cytotoxicity is simulated proportional to the current docetaxel concentration and current PCaSC and PCaC populations, i.e., δ_{S}*[DOC]*[PCaSC] and δ_{C}*[DOC]*[PCaC] (c.f. FIG. 1A), where δ_{S}<0 and δ_{C}<0 represent respectively PCaSC and PCaC DOC cytotoxicity. Total tumor regression rate (δ_{S}+δ_{C}, median 0.015 day, range 0.006-0.032) has been estimated for patients with advanced prostate cancer in a retrospective analysis of 2,353 patients from eight clinical trials, but without resolution of intratumoral heterogeneity. With PCaSC docetaxel sensitivity being about 25% of PCaC sensitivity, the individual rates of δ_{S} and δ_{C} can be determined as a function of the mathematically-predicted PCaSC proportion in the tumor at the different treatment time points to satisfy the overall tumor regression rates. DOC therapy (one bolus injection on day 1 every three weeks) can be simulated beginning at different IADT cycles (first, second, etc.). Concurrent treatment with IADT and DOC can be simulated as well as alternating therapy with DOC given in IADT off-treatment cycles. Docetaxel timing-dependent TTP can be determined for each individual patient and the patient population as a whole. Progression free survival after IADT alone and TTP simulated for IADT+DOC at different times of IADT can be visualized via Kaplan-Meier curves

(2) Summary.

This study integrates mathematical, biological, clinical and statistical expertise to test the hypothesis that PCaSC dynamics underlie response to therapy and evolution of resistance in IADT. Mathematical modeling of these response dynamics in early IADT cycles can predict responses in subsequent treatment cycles, and help identify patient-specific response dynamics as triggers to pause and resume IADT for optimal responses and maximally delayed TTP. This exploratory high-risk high-reward project produces a significant conceptual advance in PCa treatment, steering clinicians away from continuous androgen deprivation at maximum tolerable dose unit the tumor becomes resistant towards an IADT protocol, using triggers based on individual patients' response dynamics Herein, retrospective patient data from a clinical trial is stratified into model training and model validation sets. While this developmental project focuses on PCa, the concept and methodologies are translatable to multiple other cancers that become resistant to continuous MTD therapy.

### 2. Example 2: Prostate-Specific Antigen Dynamics Predict Individual Responses to Intermittent Androgen Deprivation

a) Results

(1) Mathematical Model Accurately Simulates Patient-Specific IADT Response Dynamics

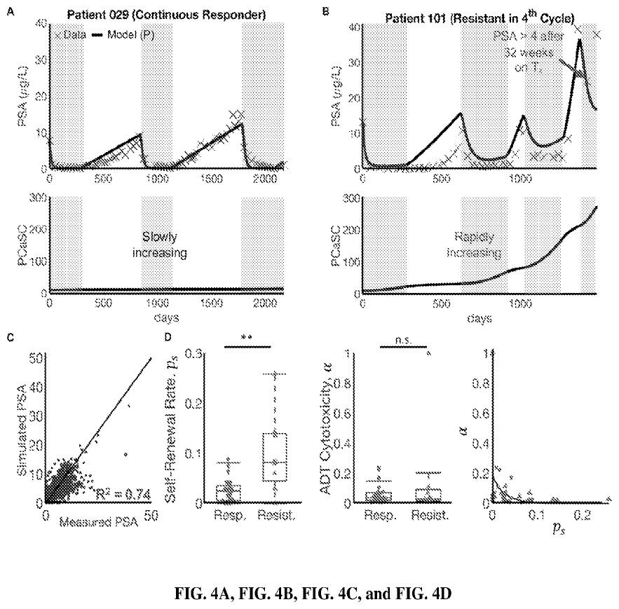

The model was calibrated to and assessed for accuracy on longitudinal data from a prospective Phase II study trial conducted in 109 men with biochemically recurrent prostate cancer treated with IADT. Stratified random sampling was used to divide the data into training and testing cohorts. Assuming that uninhibited PCaSCs divide approximately once per day, 2 (day⁻¹) was set to ln(2) and parameter estimation was used to find the remaining parameters. The model was calibrated to the training cohort data with two population-uniform parameters (PSA production rate ρ=1.87E-04 (μg/L day⁻1), decay rate φ=0.0856 (day⁻1)) and two patient-specific parameters (median PCaSC self-renewal rate pₛ=0.0278 [2.22E-14,0.2583] (non-dimensional), ADT cytotoxicity rate α=0.0360 [0.0067,1] (day⁻1)). The model results captured clinically measured longitudinal PSA dynamics of individual responsive and resistant patients (FIG. 4A, 4B) and the population as a whole (R²=0.74 FIG. 4C). The corresponding PCaSC dynamics demonstrated a rapid increase in the PCaSC population in patients that became castration resistant compared to patients that remained sensitive throughout the trial. Model simulations also showed that emergence of resistance is a result of selection for the PCaSCs during on-treatment phases. Coinciding with this transition to castration resistance, analysis of model parameters revealed that resistant patients had significantly higher PCaSC self-renewal rates than responsive patients (median pₛ=0.0249 [1.18E-13,0.0882] for responsive patients vs. pₛ=0.0820 [2.2E-14,0.2583] for resistant patients, p<0.001, FIG. 4D). As shown in FIG. 4D, analysis revealed an exponential relationship between pₛ and α and therefore these parameters are not independent. This relationship allows prediction of future cycles based on PCaSC self-renewal rate pₛ as the single, identifiable, independent patient-specific parameter.

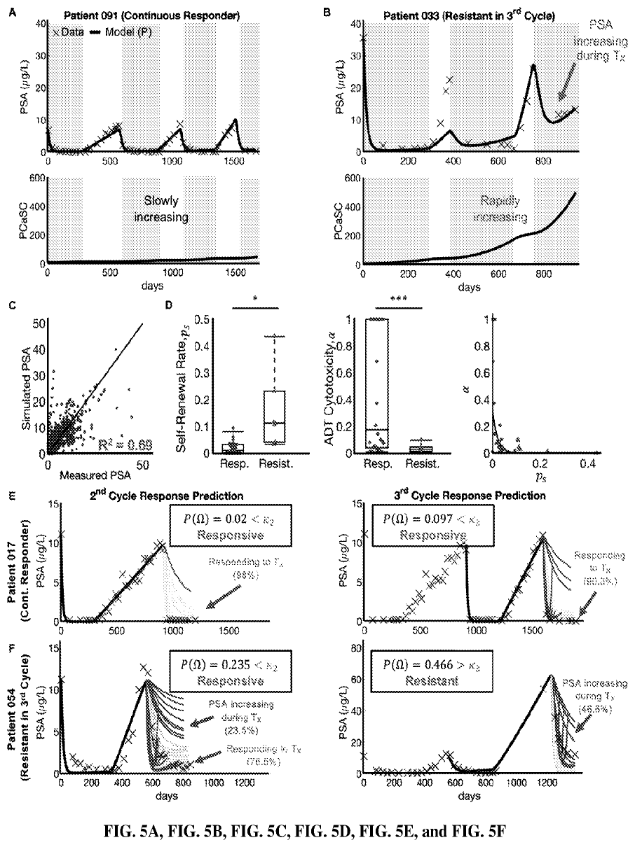

To assess the accuracy of the model, we set the PSA production rate ρ and decay rate φ to the values obtained from the training cohort and identified patient-specific values for pₛ and corresponding α in the testing cohort. With these, the model was able to fit the data equally well (R²=0.69) and the resulting parameter distributions and relationships were similar to those found in the training cohort (FIG. 5A-D).

(2) PCaSC Self-Renewal Rates Underlying Observable PSA Dynamics can Predict Subsequent IADT Cycle Responses

To predict the evolution of resistance in subsequent treatment cycles, we fit the model to single treatment cycles for patients in the training set, again setting the PSA production rate ρ and decay rate φ to the values previously found. The self-renewal and ADT cytotoxicity rates maintained the exponential relationship, previously obtained when optimizing over all IADT cycles. The distributions of the self-renewal rate pₛ and the corresponding ADT cytotoxicity rate α, as well as their relative change from cycle to cycle, were used to predict responses in subsequent cycles of patients in the training cohort (see Materials and Methods). An appropriate resistance threshold was obtained for each cycle based on the forecast simulations of the training set. Using these thresholds, model forecasting was completed on the testing set and the patient was classified as either responsive or resistant in the subsequent treatment cycle. Representative examples of second and third cycle predictions for one responsive patient and one resistant patient from the testing cohort are shown in FIG. 5E-F. Patient 017 was a continuous responder who underwent three cycles of IADT before the end of the trial. The model correctly predicted responsiveness in cycles two and three based on the parameters fitted in cycles one and two, respectively. Patient 054 became resistant in the third IADT cycle and the model was able to correctly predict response in cycle two and resistance in the third cycle based on the thresholds learned in the training set. The model yielded a sensitivity of 57% and specificity of 94% over all subsequent IADT cycles for patients in the test cohort. The overall accuracy of the model was 90%.

(3) IADT without Induction Period Significantly Increases TTP

In a study by Crook et al., continuous ADT was compared to IADT in localized PCa patients and they found that IADT was non-inferior to ADT using overall survival as the clinical endpoint. A similar study by Hussain et al. conducted in metastatic, hormone sensitive PCa patients found that neither regimen proved superior. These findings are likely the result of the 7-8 months induction period, which resulted in the competitive release of the resistant phenotype once the androgen sensitive subpopulation was eliminated. Administering IADT without such an induction period would allow sufficient time for the sensitive subpopulation to efficiently compete with the resistant subpopulation and prolong time to progression and ultimately, overall survival.

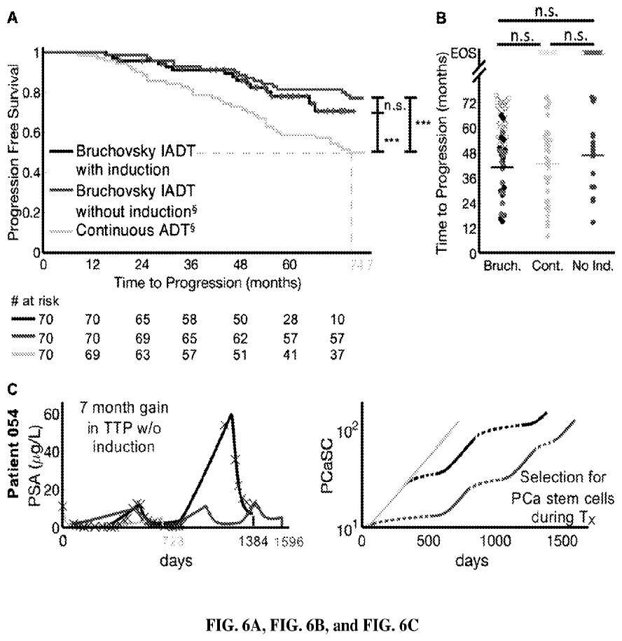

To test these hypotheses, we simulated continuous ADT and IADT without the 36-week induction period and compared predicted TTP against the trial by Bruchovsky et al. For IADT simulations, treatment was administered until PSA fell below 4 μg/L and resumed again once it rose above 10 μg/L at simulated measurements every 4 weeks. Both protocols were simulated until PSA progression or for 10 years (end of simulation, EOS), with progression defined as three sequential increases in PSA during treatment. For the IADT protocol, progression was also defined as PSA>4 μg/L at both 24 and 32 weeks on treatment, as used in the Bruchovsky trial. Comparing the TTP from the trial results against simulated continuous ADT showed that IADT with an induction period resulted in significantly longer TTP (FIG. 6A). Though median TTP was not reached for either IADT protocol, FIG. 6B shows that the average TTP was approximately 5 months longer for IADT without the induction period when compared to the actual trial data and the continuous ADT simulations. FIG. 6C shows TTP could be increased by more than 7 months in selected patients.

(4) Alternative Treatment Decision Thresholds can Further Increase TTP

In the ongoing pilot study (NCT02415621) of IADT in metastatic, castration resistant PCa patients, IADT is paused after a decline in PSA to below 50% of pre-treatment PSA levels and is resumed once PSA returns to the pre-treatment level. At the time of writing, of the 18 patients currently enrolled for more than 12 months, four have developed PSA and radiographic progression. The median time to progression has not been reached, but is at least 20 months greater than a contemporaneous cohort treated continuously with MTD until progression, as well as published cohorts. The treatment group has received an average cumulative dose of less than half that of standard of care treatment.

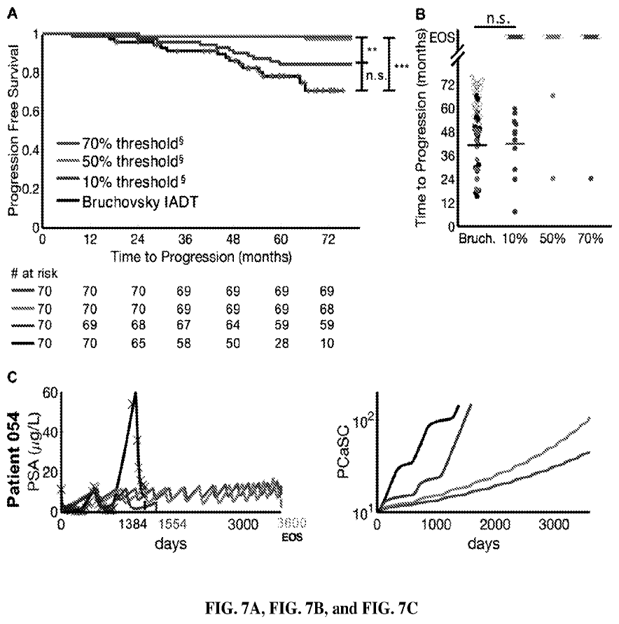

To test the validity of the model, we simulated IADT using a 50% threshold, as well as 10% and 70% and compared the results against those obtained in the Bruchovsky trial. Pausing treatment once PSA falls below 70% and 10% of the pre-treatment PSA, checking every two weeks, results in significantly longer TTP when compared to the Bruchovsky trial protocol (FIG. 7). Additionally, the average cumulative dose could be significantly reduced depending on the threshold used. With a 10% threshold, the average cumulative dose is ˜50% of that of the Bruchovsky study. A 50% threshold results in ˜22% of the cumulative dose used in the Bruchovsky study, while 70% results in ˜20%. Controlling for the total time that each patient participated in the trial results in about 30%, 15%, and 13% of the comulative dose used in the Bruchovsky study for thresholds of 10%, 50%, and 70%, respectively.

(5) Concurrent Docetaxel Administration Provides Favorable TTP

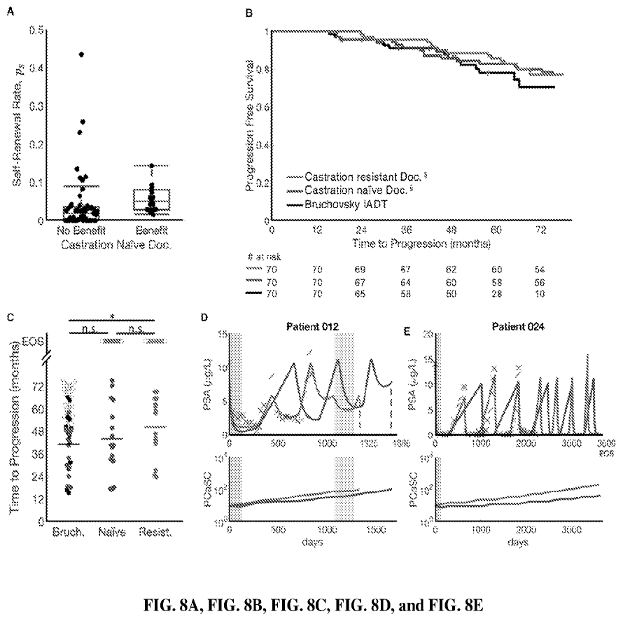

We sought to investigate the effect of docetaxel (DOC) in IADT in biochemically recurrent PCa patients by simulating six cycles of DOC with concurrent ADT, followed by IADT (as defined in the Materials and Methods section). Model analysis showed that patients with higher stem cell self-renewal rates would benefit the most from DOC in a castration naïve setting (prior to progression, FIG. 8A). Comparing progression free survival showed that castration naïve docetaxel could increase TTP (FIG. 8B). We also tested the ability of DOC to increase survival after the development of resistance to IADT. To do this, we simulated IADT, following the Bruchovsky trial protocol, and then added ten cycles of DOC plus continuous ADT after resistance developed. As shown in FIG. 8C, this increased mean TTP from 41.18 months observed in the Bruchovsky trial to model-predicted 50.14 months.

(6) First Cycle PCaSC Self-Renewal Rate Stratifies Patients Who can Benefit from Docetaxel

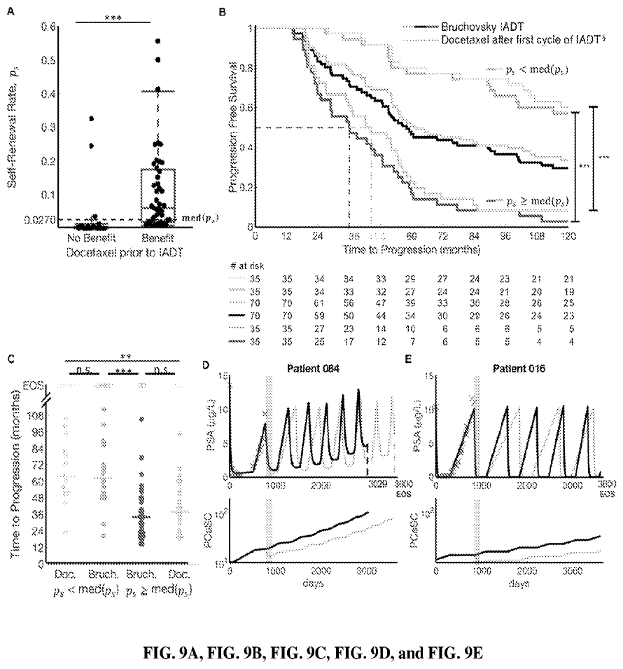

As shown above, the stem cell self-renewal rate plays a significant role in IADT and could accurately predict a patient's response in subsequent cycles after just the first cycle. With this, we used the first cycle pₛ value to simulate six cycles of DOC with concurrent ADT followed by IADT and found that patients with pₛ≥med(pₛ) could benefit from DOC after the first cycle of IADT (FIG. 9A). There was a significant difference in TTP between patients with a high pₛ and those with a low pₛ both when simulating IADT with and without DOC after the first cycle (FIG. 9B). Though the difference in TTP was not significantly higher with DOC given after the first cycle (FIG. 9C), median TTP increased from 35 to 41.5 months in those patients with pₛ≥med(pₛ), as shown in FIG. 9B.

b) Discussion

Androgen deprivation therapy is not curative for advanced prostate cancer, as patients often develop resistance. IADT is a promising approach to counteract evolutionary dynamics by reducing competitive release of the resistant subpopulation during treatment holidays. Since IADT is highly dynamic, maximum efficacy requires continuous, accurate estimates of sensitive and resistant subpopulations.

Here we present a simple mathematical model of evolutionary dynamics within biochemically recurrent prostate cancer during IADT. The model has been trained with two parameters that are uniform across all patients and only two patient-specific parameters, which are interconnected, allowing the ability to further reduce them to a single, measurable parameter for each patient. Model simulations support the central hypothesis that the evolution of PCaSCs is highly correlated with the development of resistance to IADT. Resistant patients are likely to have higher PCaSC self-renewal rates than responsive patients, leading to increased production of PCaSCs and ultimately differentiated cells, thereby accelerating PSA dynamics with each treatment cycle. The results show that that cancer stem cell self-renewal is likely to increase during prolonged treatment.

Using longitudinal PSA measurements and observed clinical outcomes from the IADT trial by Bruchovsky et al., the model was calibrated to clinical data and predicted the development of resistance with a 90% accuracy. The theoretical study produced three important clinical findings: (1) IADT outcomes in prior studies were adversely affected by the 8-month induction period, which reduced IADT overall survival comparable to that of continuous ADT. (2) With IADT, applying a PSA treatment threshold that depends on pre-treatment PSA levels (rather than a fixed value for all patients) can significantly increase TTP. (3) Early treatment response dynamics during IADT can identify patients that may potentially benefit from concurrent docetaxel treatment, particularly patients with a large stem cell self-renewal rate after one cycle of IADT.

The study also demonstrated the value of ongoing model simulations in predicting outcomes from each treatment cycle throughout the course of therapy. By accumulating data from each cycle to continuously estimate the current tumor population dynamics, model simulations could predict the response to the next cycle with a sensitivity and specificity of 57% and 94%, respectively and an overall accuracy of 90%. This ability to learn from prior treatments and predict future outcomes adds an important degree of flexibility to a cancer treatment protocol—a game theoretic strategy termed “Bellman's Principle of Optimality” that greatly increases the physician's advantage.

For those predicted to become resistant in the next cycle of IADT, an ideal model also predicts alternative treatments that could produce better clinical outcomes. The role of docetaxel in metastatic, hormone-sensitive PCa has been investigated in three studies in the past five years. The GETUG-AFU15 study found a non-significant 20% increase in overall survival in high volume disease (HVD) patients who received DOC concurrently with continuous ADT, but no survival benefit in those with low volume disease (LVD). Subsequently, the results of the STAMPEDE trial found that DOC administration resulted in a more than 12 months overall survival benefit with a median follow-up of 43 months. Finally, the CHAARTED trial showed a statistically significant overall survival benefit from adding DOC in patients with HVD; however, no statistically significant survival benefit was found in LVD patients.

Here we explored the option of adding docetaxel in the treatment of biochemically recurrent PCa. We found that estimating the PCaSC self-renewal rate pₛ using data from the first cycle of IADT could stratify patients who would receive the most benefit from concurrent administration of docetaxel. These results emphasize the critical heterogeneity within patients that affect response to therapy and the important role of quantitative models in identifying patient-specific parameters and defining appropriate treatment protocols based on model predictions.

The study has some limitations in both the clinical data set and quantitative models. Since only 21% of the 70 patients progressed before the conclusion of the trial, the clinical data included more responsive than resistant patients in both the training and testing

cohorts. Additionally, the majority of patients progressed within their second or third cycle, with only 24 patients entering the fourth cycle. The scarcity of available data during the fourth cycle made finding an appropriate resistance threshold challenging. A larger training data set increases the sensitivity and overall accuracy of the model.

A limitation of the quantitative model is the use of dynamic PSA values as the sole biomarker of PCa progression. PCa can become aggressive and metastatic despite low levels of serum PSA with development of androgen independent prostate cancer, most notably neuroendocrine prostate cancer. Additional serum biomarkers such as circulating tumor cells (CTCs) and cell-free DNA (cfDNA) may prove useful in estimating intratumoral evolutionary dynamics in subsequent trials. With the CellSearch platform, higher CTCs enumeration >5 cells per 7.5 mL of peripheral blood has been shown to be prognostic and portend worse overall survival in metastatic CRPC patients; however detecting CTCs in biochemically recurrent patients has been labor-intensive with low yield.

In conclusion, the study demonstrates that a simple mathematical model based on cellular dynamics in prostate cancer can have a high predictive power in a retrospective data set from patients with biochemically recurrent PCa undergoing IADT. In particular, we demonstrate the model can use data from each treatment cycle to estimate intratumoral subpopulations and accurately predict the outcome of subsequent cycles. Furthermore, in patients who are predicted to fail therapy in the next cycle, alternative treatments for which a response is more likely can be predicted. We conclude that PSA dynamics can prospectively predict treatment response to IADT, indicating ways to adapt treatment to delay TTP.

c) Materials and Methods

(1) IADT Clinical Trial Data

The Bruchovsky prospective Phase II study trial was conducted in 109 men with biochemically recurrent prostate cancer. IADT consisted of 4 weeks of Androcur as lead-in therapy, followed by a combination of Lupron and Androcur, for a total of 36 weeks. Treatment was paused if PSA has normalized (<4 μg/L) at both 24 and 32 weeks, and resumed when PSA increased above 10 μg/L. PSA was measured every four weeks. Patients whose PSA had not normalized after both 24 and 32 weeks of being on treatment were classified as resistant and taken off of the study. We analyzed the data of 79 patients who had completed more than one IADT cycle. One patient was omitted for inconsistent treatment, seven were omitted due to the development of metastasis and/or local progression, and one was omitted due to taking multiple medications throughout the trial, resulting in 70 patients included in the analysis. To calibrate and assess the accuracy of our model, the data was divided into training (n=35, 27 responsive, 8 resistant) and testing (n=35, 28 responsive, 7 resistant) cohorts, respectively matched for clinical response to treatment.

(2) Mathematical Model of IADT Response

We developed a mathematical model of PCaSC (S), non-stem (differentiated) cells (D), and serum PSA concentration (P). PCaSCs divide with rate λ (day⁻¹) to produce either a PCaSC and a non-stem PCa cell with rate 1−pₛ (asymmetric division) or two PCaSCs at rate pₛ (symmetric division) with negative feedback from differentiated cells (15). Differentiated cells exclusively produce PSA at rate μ (μg/L day⁻1), which decays at rate φ (day⁻¹). Unlike androgen-independent PCaSCs, differentiated cells die in response to androgen removal at rate α (day⁻¹) (33). IADT on and off cycles are described with parameter Tₓ, where Tₓ=1 when IADT is given and Tₓ=0 during treatment holidays.

The coupled mathematical equations describing these interactions are shown below.

$$dt \\ \frac{dS}{dt} = ( \frac{S}{S + D} ) {p}_{s} λ S , \frac{dD}{dt} ( 1 - \frac{S}{S + D} ) λ S - α {T}_{x} D , \frac{dP}{dt} ρ D - φ P \qquad ( 1 )$$

(3) Mathematical Model Training and Validation

We assume uninhibited PCaSCs divide on average once per day and set λ=ln(2). To reduce model complexity and prediction uncertainty, we assume PSA production rate (ρ) and decay rate (φ) to be uniform between patients. PCaSC self-renewal rate (pₛ) and differentiated cell ADT sensitivity (α) are correlated and assumed to be patient-specific. We used particle swarming optimization (PSO) to identify population uniform and patient-specific model parameters that minimize the least squares error between model simulation and patient data in the training cohort. The trained mathematical model is assessed for accuracy in the validation cohort. The learned population uniform parameters are kept constant for all patients, and PSO is performed to find appropriate values for pₛ and a to produce accurate data fits.

(4) Adaptive Bayesian Response Prediction

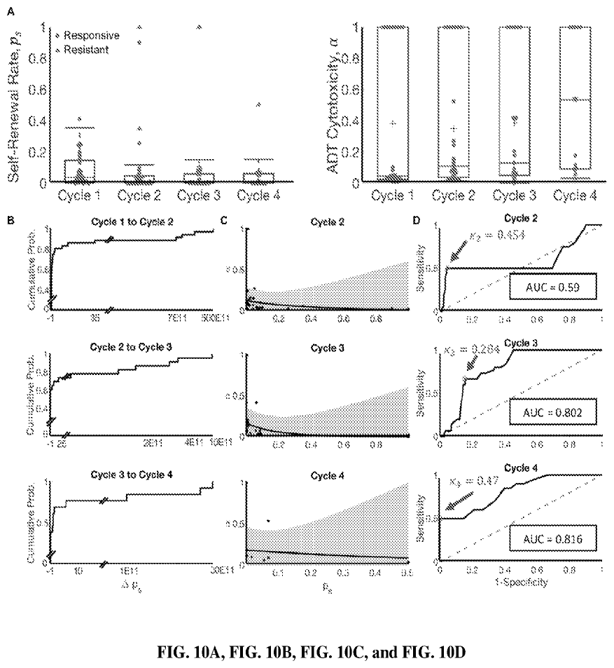

In order to predict the evolution of resistance, we started by fitting the model to each cycle of the training cohort data individually. That is, finding the optimal values of pₛ and α, while allowing φ and ρ to remain fixed at the values previously found, to fit one cycle of data at a time (FIG. 10A). We then measured the relative change in pₛ between cycles and used this to generate cumulative probability distributions as shown in FIG. 10B. Sampling from the 95% confidence interval around the exponential curve relating pₛ to α in cycle i+1 (FIG. 10C), we found a corresponding αᵢ₊₁. This p_{s,i+1} and αᵢ₊₁ were used to predict the response in cycle i+1. This process was repeated 1000 times to generate 1000 predictions. In line earlier trials, resistance was defined as PSA increasing during treatment and/or a PSA level above 4 μg/L at both 24 and 32 weeks after the start of a cycle of treatment. Simulations that satisfied either of these conditions were classified as resistant. Thus, for each cycle prediction we obtained a probability of resistance (P(Ω)=number of resistance predictions out of 1000 simulations).

To determine whether to categorize a patient as responsive or resistant based on our predictions, we used the results from the training cohort data to find a cycle-specific threshold κᵢ for each cycle (FIG. 10D). If P(Ω)>κᵢ, then the patient was predicted as resistant in cycle i. For each cycle, a value of κ was chosen that would maximize the sensitivity (predicting resistance when a patient is indeed resistant), and specificity (predicting resistance when a patient is actually responsive) of the training cohort. Each resistance threshold was used to predict response or resistance in the training cohort.

(5) Modeling Concurrent Docetaxel

Unlike ADT, docetaxel can induce cell death in both PCaSCs and non-stem cells, though to a lesser degree in PCaSCs compared to non-stem cells (35). To model this, we extended the current model to include death of each cell type at rates δ=(day⁻¹) and δ>(day⁻¹) That is,

$$\frac{dS}{dt} = ( \frac{S}{S + D} ) {p}_{s} λ S - {δ}_{S} {T}_{xD} S , \\ \frac{dD}{dt} = ( 1 - \frac{S}{S + D} {p}_{s} ) λ S + α {T}_{x} D - {δ}_{D} {T}_{xD} S , \qquad ( 2 )$$

where T_{xD}=1 when docetaxel is on and T_{xD}=0 otherwise. Each cycle of docetaxel was simulated as a single dose on day n (T_{xD}=1) followed by three weeks without docetaxel (T_{xD}=0). The parameters δ₌=0.0027 and δ_{>}=0.008 were chosen such that approximately three times more non-stem cells died than PCaSCs.

### 3. Example 3: Clinical Decision Support Tool for Prostate Cancer Adaptive Abiraterone Treatment

a) Materials and Methods

(1) Adaptive Abiraterone Trial Data

The adaptive therapy trial was conducted in 18 metastatic castration resistant prostate cancer (mCRPC) patients. Prior to trial registration, patients received abiraterone plus prednisone as standard of care. Patients who achieved a 50% or more decline of their PSA were eligible to enroll in the trial. Each patient's PSA immediately prior to beginning abiraterone was considered his baseline PSA. Abiraterone was stopped after trial enrollment and resumed once PSA rose above pre-abiraterone baseline level. Therapy was stopped again once PSA fell below 50% of pre-treatment PSA. PSA was monitored every 4-6 weeks, with restaging bone scan, pelvic and abdominal CT scan performed every 12 weeks. Patients remained on the trial until radiographic progression based on PCWG2 criteria.

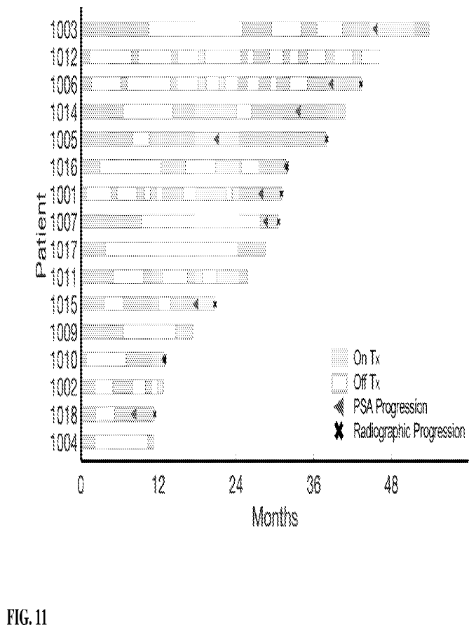

123. We received and analyzed longitudinal PSA data for 16 of the 18 patients enrolled in the trial. Due to the nature of the mathematical model, we assessed the patients based on PSA progression, rather than radiographic progression. PSA progression is defined as PSA increasing ≥25% and at least 2 ng/mL above the nadir, confirmed by a second value 3 or more weeks later. Eight of the 16 patients developed PSA progression within the first four cycles of treatment. The treatment times for the 16 patients included in the analysis are shown in FIG. 11.

(2) Mathematical Model

The mathematical model is adapted from the work. The model incorporates PCaSCs (S), non-stem differentiated cells (D), and PSA (P) interactions. PCaSCs divide at rate λ (day⁻¹) to produce either a PCaSC and non-stem PCa cell with probability 1−pₛ (asymmetric division) or two PCaSCs with probability pₛ (symmetric division), with negative feedback from the differentiated cells. The differentiated cells die at rate α in response to treatment, which is modulated by the parameter Tₓ=1 and Tₓ=0, denoting when treatment is on and off, respectively. PSA is produced by the differentiated cells at rate ρ (μg/L day⁻¹) and decays at rate φ(day⁻¹). The mathematical equations describing these interactions are given by

$$dt \\ \frac{dS}{dt} = ( \frac{S}{S + D} ) {p}_{s} λ S , \frac{dD}{dt} ( 1 - \frac{S}{S + D} ) λ S - α {T}_{x} D , \frac{dP}{dt} ρ D - φ P . \qquad ( 1 )$$

(3) Model Calibration and Validation

Analysis has shown that uninhibited PCaSCs divide once a day at rate λ=ln(2) Sensitivity and correlation analysis showed that p and could be uniform among all patients and pₛ and α be patient-specific without significantly changing the model results. As such, we used a leave-one-out analysis to determine the uniform values for ρ and φ, while allowing pₛ and α to be patient-specific. That is, for patient j, we used nested optimization to find the uniform values for and ρⱼ and φⱼ the patient-specific values for pₛ and α for patients {1, 2, . . . j−1, j+1, . . . 18} in the training set. We then validated the model using ρᵢ and φᵢ to determine the patient-specific pₛ and α for patient j. This process was repeated for all 18 patients.

(4) Treatment Response Prediction

For patient pᵢ and φⱼ were used to fit the model to each cycle individually for all patients in the training set. That is, finding the optimal value for pₛ and α while allowing pⱼ and φⱼ to remain fixed for each individual cycle. Given pₛ and α for cycle i for patient j, we used the relative changes in pₛ for all patients in the training set to generate the cumulative probability distribution of relative changes in pₛ from cycler to cycle i+1. We sampled from this distribution to determine 100 values of pₛ for cycle i+1. The work demonstrated that pₛ and α are exponentially related. To determine the corresponding αₜ₊₁ values, we sampled from the 95% confidence interval around the exponential curve relating pₛ and α to find 100 values for αᵢ₊₁. We used these values to predict patient j's response in cycle i+1. More details of the prediction process can be found in Brady-Nicholls et al.

Each model simulation was determined to be responsive or resistant based on the PSA progression criteria defined in the trial. That is, if PSA increased more than 25% and at least 2 above the nadir during treatment, then the simulation was classified as resistant. Of the 100 response simulations, we quantified the number of resistant simulations to derive a probability of resistance Pr(Ω). If the Pr(Ω) was greater than a given threshold (obtained from the training set), then the prediction was considered resistant. Otherwise, it was classified as responsive.

b) Results

(1) Model Accurately Describes Clinical Data

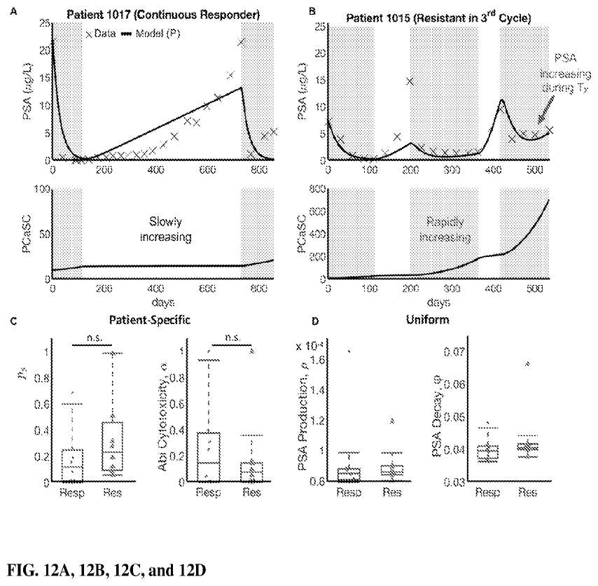

We demonstrated that the model is able to accurately fit longitudinal data from biochemically recurrent patients receiving intermittent ADT. Despite abiraterone working through an alternative mechanism to kill cancer cells⁸, the model is able to accurately describe the dynamics in the mCRPC patients (FIG. 12A-B). Additionally, in line with the results, the model fits to the data demonstrate that continuous responders have a slowly increasing PCaSC population (FIG. 12A), while patients who progress have a rapidly increasing PCaSC population (FIG. 12B). This shows that stem cell enrichment is still a plausible driver of resistance. A comparison of the stem cell self-renewal rate p_{z} between responsive and resistant patients showed that resistant patients tend to have a larger pₛ value, though not significant (FIG. 12C). The uniform values for ρ and φ for each leave-one-out analysis are shown in FIG. 12D.

(2) Early Treatment Dynamics can be Used to Predict Subsequent Response

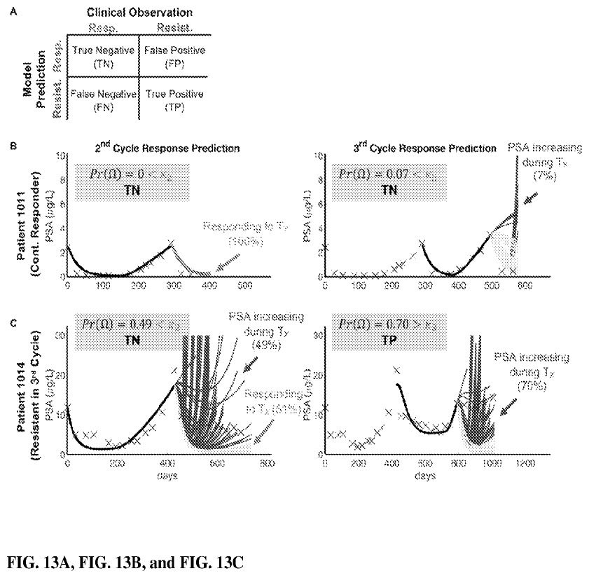

For each leave-one-out, an optimal cutoff value kᵢ for cycle i was determined to be the threshold that maximized the accuracy within the training set. Using this, Pr(Ω) was evaluated for each patient and the prediction was classified accordingly. Model predictions correctly classifying clinically observed responders as responders (Pr(Ω)<κᵢ) were denoted true negative, while correctly classified clinically resistors (Pr(Ω)>κᵢ) were denoted as true positive. The remaining classifications are shown in FIG. 13A.

The model predictions for a continuous responder and a patient who progressed in the third cycle are shown in FIG. 13B-C. The model was fit over one cycle using the ρ and φ values obtained from the training set. Using the relative change in p_{z} from cycle i to cycle i+1 as well as the exponential relationship between p_{z} and α, we obtained 100 parameter pairs of (pₛ·α) that were used to forecast the patient's response in the subsequent cycle. The model accurately predicted that Patient 1011 would continue to respond in both the second and third cycles. That is, the P_{I}(Ω) is less than the determined κ_{z} and κ_{σ} for cycles 2 and 3, respectively (FIG. 13B). Patient 1014 developed PSA progression in his third cycle of treatment. Though Pr(Ω)=0.49 in cycle 2, this is below the threshold κ_{Z} and consequently the model correctly predicts that this patient will continue to respond. In cycle 3, Pr(Ω)=0.70 which is larger than κ₃. This is a correct prediction as Patient 1014 develops resistance in cycle 3. Overall, the model is able to correctly predict patient response with 78% accuracy (specificity=92%, sensitivity=38%).

(3) Incorporating Metastatic Burden

Despite treatment, several patients continued to develop metastases during the trial. Though rising PSA has been shown to be correlated with metastatic burden, it is not predictive of the development of new metastatic growths. We sought to investigate whether correlating metastatic growth with the driver of treatment resistance, the stem cell self-renewal rate pₛ, can be used to improve the accuracy of the model.

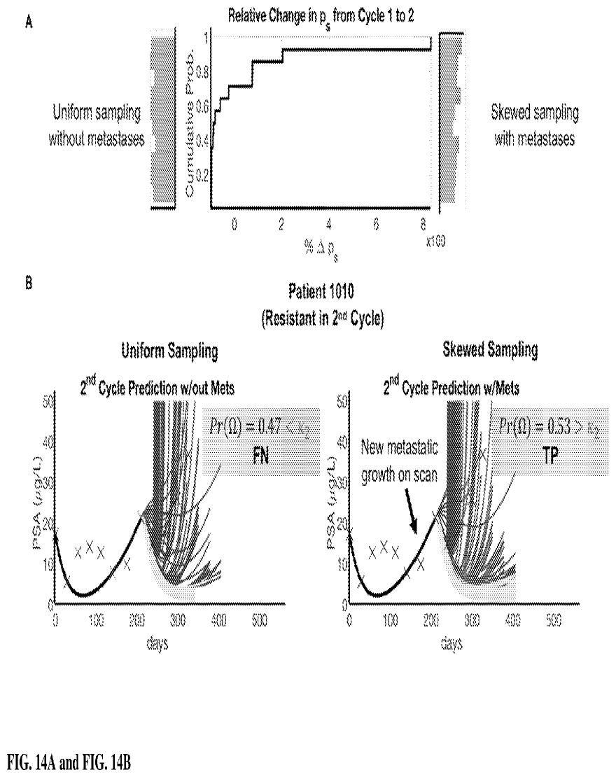

As described, (p_{z},α) pairs for cycle i+1 were obtained by uniformly sampling from the cumulative probability distribution of relative changes in p_{z} from cycle i to i+1. FIG. 14A shows that in general, the relative change ranged between −75% and 150%, with about 30% of the patients experiencing larger relative changes in p. To incorporate the metastatic burden, we used a skewed sampling to sample from the cumulative probability distribution, with the degree of the skew dependent on the number of new metastatic growths. This resulted in larger relative changes in pₛ. As shown in FIG. 12B, larger pₛ values translated into earlier treatment progression. Consequently, the skewed sampling results in more resistant simulations, thereby increasing the Pr(Ω) It should be noted that this also resulted in κᵢ changing in response to larger Pr(Ω) for particular patients.

A comparison between the model predictions using a uniform and skewed sampling for an individual patient are shown in FIG. 14B. Patient 1010 developed a new metastatic growth during his first cycle of treatment. With a uniform sampling, the model predicted that the Pr(Ω)=0.47. As this was below the threshold κ₂, the model incorrectly predicted that this patient would continue to respond in the second cycle (false negative). Using a skewed sampling, the Pr(Ω) increased to 0.53, which was larger than κ₂ resulting in a correct resistant prediction (true positive). Overall, incorporating each patient's metastatic increased the accuracy to 81% (specificity=92%, sensitivity=50%).

c) Discussion

In this study, we have extended the simple model of PCaSC and PSA dynamics to predict mCRPC patient responses to adaptive abiraterone. The model has been calibrated and validated against clinical data from 16 mCRPC patients from a trial.

With just two patient-specific parameters, the results show that the model is able to accurately describe individual patient dynamics We found that stem cell enrichment is a likely driver of treatment resistance, despite abiraterone working through an alternative pathway than ADT. Despite that using the established prediction process, the model was able to use early treatment evolutionary dynamics to predict subsequent response with 78% accuracy.

Incorporating the metastatic burden improved the overall accuracy to 82%, with a specificity of 92% and a sensitivity of 50%. We note that though this increased the sensitivity of the model, this is significantly lower than the 73% sensitivity obtained in another study. This is likely due to the few progression events within this patient cohort. This limitation made it difficult to determine the optimal threshold value for cycle predictions where few patients progressed, resulting in a higher proportion of false positive results. We are confident that the model accuracy, as well as sensitivity and specificity, has the potential to increase with a larger patient cohort. Nevertheless, the patients who were incorrectly predicted to develop resistance during the second cycle instead progressed during the third cycle. As such, the model serves as an early indicator of treatment resistance for a subset of patients.

To conclude, this validation study demonstrates the utility of mathematical modeling in clinical decisions. Using a simple dynamic model of PCaSC, non-stem cells, and PSA, we are able to predict who may or may not respond to subsequent treatment. This offers clinicians with an additional decision support tool to use when deciding how to effectively treat patients, while minimizing adverse events and toxicity and maximizing survival.

### 4. Example 4

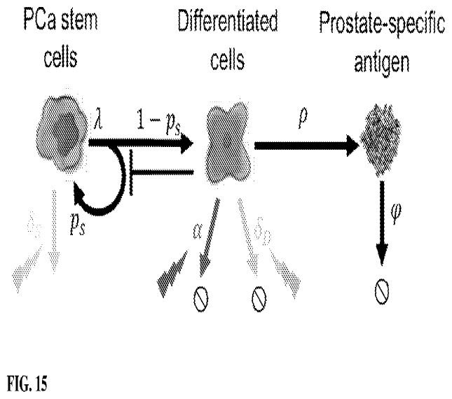

We also used the mathematical to investigate the optimal time to administer chemotherapy in metastatic prostate cancer patients. Unlike androgen deprivation therapy (ADT), chemotherapy can induce cell death in both PCaSCs and differentiated cells, though to a lesser degree in PCaSCs compared to differentiated cells. To model this, we extended the model to include death of each cell type at rates δ [symbol] and δ [symbol] (FIG. 15).

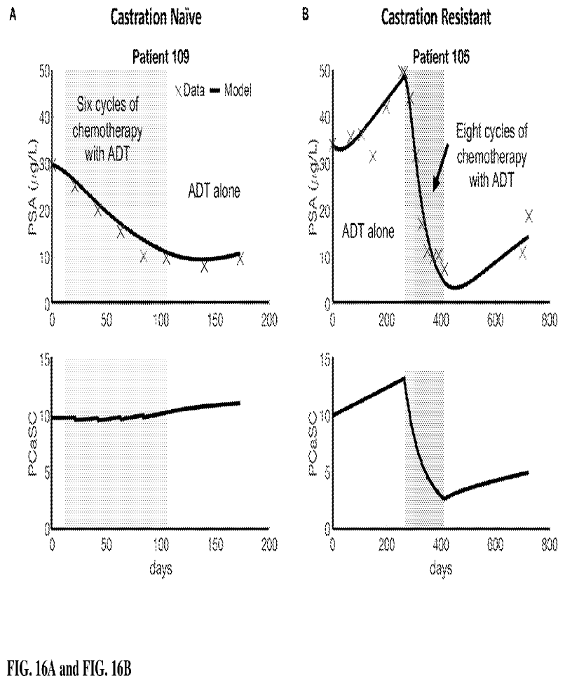

The model was calibrated to PSA data from 56 castration naïve patients, that is they received up to six cycles of concurrent chemotherapy and ADT at the start of therapy, followed by ADT alone and 38 castration resistant patients who received up to 10 cycles of concurrent therapy after developing resistance to ADT alone. The castration resistant patients received follow-up ADT after concurrent treatment. The model was able to fit each set of patients equally well (FIG. 16).

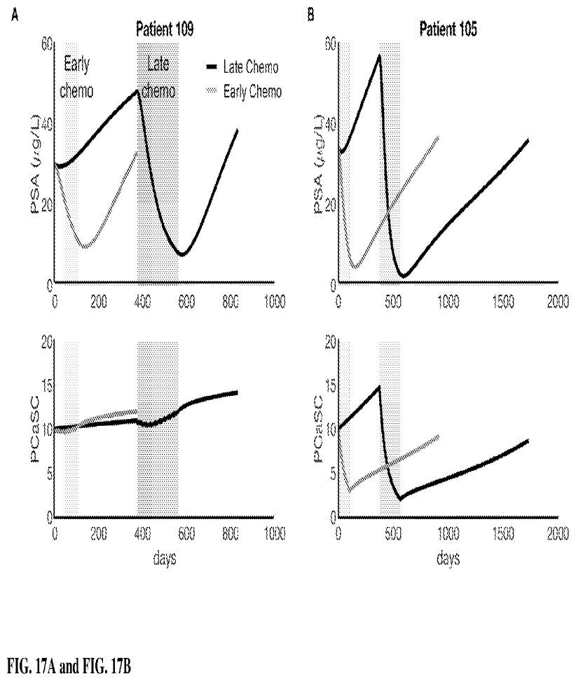

After calibrating and validating the model, we used the optimal parameter values to simulate administering concurrent therapy either before or after ADT (FIG. 17). Model simulations show that the optimal time of concurrent therapy is patient-specific and can be determined using early treatment dynamics, offering another tool for clinicians to use when determining how and when to treat patients.

### D. REFERENCES

Anderson A R A, Quaranta V. Integrative mathematical oncology. Nat. Rev. Cancer. 2008 March; 8(3):227-34.
Basanta D, Gatenby R A, Anderson A R A. Exploiting evolution to treat drug resistance: combination therapy and the double bind. Mol. Pharm. 2012 Apr. 2; 9(4):914-21. PMCID: PMC3325107
Bishop J L, Davies A, Ketola K, Zoubeidi A. Regulation of tumor cell plasticity by the androgen receptor in prostate cancer. Endocr. Relat. Cancer. BioScientifica; 2015 June; 22(3):R165-82.
Bora-Singhal N, Nguyen J, Schaal C, Perumal D, Singh S, Coppola D, et al. YAP1 Regulates OCT4 Activity and SOX2 Expression to Facilitate Self-Renewal and Vascular Mimicry of Stem-Like Cells. Stem Cells. 2015 June; 33(6):1705-18. PMCID: PMC4441573
Brady-Nicholls R, et al. Prostate-specific antigen dynamics predict individual responses to intermittent androgen deprivation. Nat Commun 11, 1750 (2020).
Bruchovsky N, Klotz L, Crook J, Malone S, Ludgate C, Morris W J, et al. Final results of the Canadian prospective phase II trial of intermittent androgen suppression for men in biochemical recurrence after radiotherapy for locally advanced prostate cancer: clinical parameters. Cancer. Wiley Subscription Services, Inc., A Wiley Company; 2006 Jul. 15; 107(2):389-95.
Chen W, Chen R, Li J, Fu Y, Yang L, Su H, et al. Pharmacokinetic/Pharmacodynamic Modeling of Schedule-Dependent Interaction between Docetaxel and Cabozantinib in Human Prostate Cancer Xenograft Models. J. Pharmacol. Exp. Ther. American Society for Pharmacology and Experimental Therapeutics; 2018 January; 364(1):13-25.
Collins A T, Berry P A, Hyde C, Stower M J, Maitland N J. Prospective identification of tumorigenic prostate cancer stem cells. Cancer Res. American Association for Cancer Research; 2005 Dec. 1; 65(23):10946-51.
Cook L M, Araujo A, Pow-Sang J M, Budzevich M M, Basanta D, Lynch C C. Predictive computational modeling to define effective treatment strategies for bone metastatic prostate cancer. Sci Rep. 2016 Jul. 14; 6(1):29384. PMCID: PMC4944130
Crook J M, O'Callaghan C J, Duncan G, Dearnaley D P, Higano C S, Horwitz E M, et al. Intermittent androgen suppression for rising PSA level after radiotherapy. N. Engl. J. Med. 2012 Sep. 6; 367(10):895-903. PMCID: PMC3521033
Dagmara Jaworska, Ewelina Szliszka. Targeting Apoptotic Activity Against Prostate Cancer Stem Cells. HMS. Multidisciplinary Digital Publishing Institute; 2017 August; 18(8):1648-21.
Damodaran S, Kyriakopoulos C E, Jarrard D F. Newly Diagnosed Metastatic Prostate Cancer: Has the Paradigm Changed? Urol Clin North Am 44, 611-621 (2017).
de Bono J S, et al. Abiraterone and increased survival in metastatic prostate cancer. N Engl J Med 364, 1995-2005 (2011).
Donovan K A, Gonzalez B D, Nelson A M, Fishman M N, Zachariah B, Jacobsen P B. Effect of androgen deprivation therapy on sexual function and bother in men with prostate cancer: A controlled comparison. Psychooncology. 2017 May 30; 27(Supp 2A):603. PMCID: PMC5709275
Donovan K A, Walker L M, Wassersug R J, Thompson L M A, Robinson J W. Psychological effects of androgen-deprivation therapy on men with prostate cancer and their partners. Cancer. 5 ed. 2015 Dec. 15; 121(24):4286-99.
Eikenberry S E, Nagy J D, Kuang Y. The evolutionary impact of androgen levels on prostate cancer in a multi-scale mathematical model. Biol. Direct. BioMed Central Ltd; 2010; 5(1):24. PMCID: PMC2885348
Enriquez-Navas P M, Wojtkowiak J W, Gatenby R A. Application of Evolutionary Principles to Cancer Therapy. Cancer Res. American Association for Cancer Research; 2015 Nov. 15; 75(22):4675-80. PMCID: PMC4693617
Fizazi K, et al. LATITUDE: A phase III, double-blind, randomized trial of androgen deprivation therapy with abiraterone acetate plus prednisone or placebos in newly diagnosed high-risk metastatic hormone-naive prostate cancer. J Clin Oncol 35, (2017).
Gallaher J, Cook L M, Gupta S, Araujo A, Dhillon J, Park J Y, et al. Improving treatment strategies for patients with metastatic castrate resistant prostate cancer through personalized computational modeling. Clin. Exp. Metastasis. Springer Netherlands; 2014 December; 31(8):991-9. PMCID: PMC5399888
Gao X, McDonald J T, Hlatky L, Enderling H. Acute and fractionated irradiation differentially modulate glioma stem cell division kinetics. Cancer Res. 2013 Mar. 1; 73(5):1481-90. PMCID: PMC3594421
Gatenby R A, Brown J, Vincent T. Lessons from applied ecology: cancer control using an evolutionary double bind. Cancer Res. American Association for Cancer Research; 2009 Oct. 1; 69(19):7499-502.
Gatenby R A, Brown J. The Evolution and Ecology of Resistance in Cancer Therapy. Cold Spring Harb Perspect Med. 2017 Jul. 14; a033415.
Gatenby R A, Maini P K. Mathematical oncology: cancer summed up. Nature. Nature Publishing Group; 2003 Jan. 23; 421(6921):321-1.
Gatenby R A. A change of strategy in the war on cancer. Nature. 2009 May 28; 459(7246):508-9.
Gli1-Mediated Regulation of Sox2 Facilitates Self-Renewal of Stem-Like Cells and Confers Resistance to EGFR Inhibitors in Non-Small Cell Lung Cancer. Neoplasia. 2015 July; 17(7):538-51.
Harris W P, Mostaghel E A, Nelson P S, Montgomery B. Androgen deprivation therapy: progress in understanding mechanisms of resistance and optimizing androgen depletion. Nat Clin Pract Urol 6, 76-85 (2009).
Higano C S. Side effects of androgen deprivation therapy: monitoring and minimizing toxicity. Urology. 2003 February; 61(2 Suppl 1):32-8.
Hoyle A P, et al. Abiraterone in “High-” and “Low-risk” Metastatic Hormone-sensitive Prostate Cancer. Eur Urol 76, 719-728 (2019).
Hussain M, Tangen C M, Berry D L, Higano C S, Crawford E D, Liu G, et al. Intermittent versus continuous androgen deprivation in prostate cancer. N. Engl. J. Med. 2013 Apr. 4; 368(14):1314-25. PMCID: PMC3682658
James N D, Sydes M R, Clarke N W, Mason M D, Dearnaley D P, Spears M R, et al. Addition of docetaxel, zoledronic acid, or both to first-line long-term hormone therapy in prostate cancer (STAMPEDE): survival results from an adaptive, multiarm, multistage, platform randomised controlled trial. Lancet. 2016 Mar. 19; 387(10024):1163-77. PMCID: PMC4800035
Jilkine A, Gutenkunst R N. Effect of dedifferentiation on time to mutation acquisition in stem cell-driven cancers. Wang E, editor. PLoS Comput. Biol. 2014 March; 10(3):e1003481. PMCID: PMC3945168
Kasper S. Identification, characterization, and biological relevance of prostate cancer stem cells from clinical specimens. Urol. Oncol. 2009 May; 27(3):301-3. PMCID: PMC2799332
Kenmotsu H, Tanigawara Y. Pharmacokinetics, dynamics and toxicity of docetaxel: Why the Japanese dose differs from the Western dose. Cancer Sci. 2015 May; 106(5):497-504. PMCID: PMC4452149
Lander A D, Gokoffski K K, Wan F Y M, Nie Q, Calof A L. Cell lineages and the logic of proliferative control. Stevens C F, editor. PLoS Biol. Public Library of Science; 2009 Jan. 20; 7(1):e15. PMCID: PMC2628408
Leder K, Pitter K, Laplant Q, Hambardzumyan D, Ross B D, Chan T A, et al. Mathematical modeling of PDGF-driven glioblastoma reveals optimized radiation dosing schedules. Cell. 2014 Jan. 30; 156(3):603-16. PMCID: PMC3923371
Lewin T, Kim J, Latifi K, Poleszczuk J, Bull J, Byrne H M, et al. Proliferation saturation index predicts oropharyngeal squamous cell cancer gross tumor volume reduction to prospectively identify patients for adaptive radiation therapy. International Journal of Radiation Oncology*Biology*Physics. 2016; 94(4):903.
Lo W-C, Chou C-S, Gokoffski K, Wan F, Lander A, Calof A, et al. Feedback regulation in multistage cell lineages. Math Biosci Eng. 2009 January; 6(1):59-82.
Morken J D, Packer A, Everett R A, Nagy J D, Kuang Y. Mechanisms of resistance to intermittent androgen deprivation in patients with prostate cancer identified by a novel computational method. Cancer Res. 2014 Jul. 15; 74(14):3673-83.
Morote J, Orsola A, Abascal J M, Planas J, Trilla E, Raventos C X, et al. Bone mineral density changes in patients with prostate cancer during the first 2 years of androgen suppression. J. Urol. 2006 May; 175(5): 1679-83—discussion1683.
Mu P, Zhang Z, Benelli M, Karthaus W R, Hoover E, Chen C-C, et al. SOX2 promotes lineage plasticity and antiandrogen resistance in TP53- and RB1-deficient prostate cancer. Science. American Association for the Advancement of Science; 2017 Jan. 6; 355(6320):84-8. PMCID: PMC5247742
Poleszczuk J T, Johnstone P A, Enderling H. Stratifying prostate cancer patients by relative lymph node involvement: population- and modeling-based study. Cancer Med. 2016 May 26; 5(8):1850-5. PMCID: PMC4884636
Portz T, Kuang Y, Nagy J D. A clinical data validated mathematical model of prostate cancer growth under intermittent androgen suppression therapy. AIP Advances. 2012.
Prokopiou S, Moros E G, Poleszczuk J, Caudell J, Torres-Roca J F, Latifi K, et al. A proliferation saturation index to predict radiation response and personalize radiotherapy fractionation. Radiat Oncol. 2015; 10(1):159. PMCID: PMC4521490
Robin X, Turck N, Hainard A, Tiberti N, Lisacek F, Sanchez J-C, et al. pROC: an open-source package for R and S+ to analyze and compare ROC curves. BMC Bioinformatics. BioMed Central; 2011 Mar. 17; 12(1):77. PMCID:
Rodriguez-Brenes I A, Komarova N L, Wodarz D. Evolutionary dynamics of feedback escape and the development of stem-cell-driven cancers. Proc. Natl. Acad. Sci. U.S.A. National Acad Sciences; 2011 Nov. 22; 108(47):18983-8. PMCID: PMC3223454
Ryan C J, et al. Abiraterone in metastatic prostate cancer without previous chemotherapy. N Engl J Med 368, 138-148 (2013).
Siegel R L, Miller K D, Jemal A. Cancer statistics, 2017. CA Cancer J Clin. 3rd ed. 2017 Jan. 5; 67(1):7-30.
Sweeney C J, Chen Y-H, Carducci M, Liu G, Jarrard D F, Eisenberger M, et al. Chemohormonal Therapy in Metastatic Hormone-Sensitive Prostate Cancer. N. Engl. J. Med. 2015 Aug. 20; 373(8):737-46.
Tannock I F, de Wit R, Berry W R, Horti J, Pluzanska A, Chi K N, et al. Docetaxel plus prednisone or mitoxantrone plus prednisone for advanced prostate cancer. N. Engl. J. Med. 2004 Oct. 7; 351(15):1502-12.
Tariq I, Chen T, Kirkby N F, Jena R. Modelling and Bayesian adaptive prediction of individual patients' tumour volume change during radiotherapy. Phys Med Biol. IOP Publishing; 2017 Jan. 31; 61(5):2145-61.
Thomsen F B, et al. Prediction of metastatic prostate cancer by prostate-specific antigen in combination with T stage and Gleason Grade: Nationwide, population-based register study. PLoS One 15, e0228447 (2020).
Visvader J E, Lindeman G J. Cancer stem cells in solid tumours: accumulating evidence and unresolved questions. Nat. Rev. Cancer. 2008 October; 8(10):755-68.
Wilkerson J, Abdallah K, Hugh-Jones C, Curt G, Rothenberg M, Simantov R, et al. Estimation of tumour regression and growth rates during treatment in patients with advanced prostate cancer: a retrospective analysis. Lancet Oncol. 2017 January; 18(1):143-54.
Yankeelov T E, Quaranta V, Evans K J, Rericha E C. Toward a Science of Tumor Forecasting for Clinical Oncology. Cancer Res. American Association for Cancer Research; 2015 Jan. 15; 75(6):918-23. PMCID: PMC4359948
You L, Brown J S, Thuijsman F, Cunningham J J, Gatenby R A, Zhang J, et al. Spatial vs. non-spatial eco-evolutionary dynamics in a tumor growth model. J. Theor. Biol. 2017 Dec. 21; 435:78-97.
Yvon A M, Wadsworth P, Jordan M A. Taxol suppresses dynamics of individual microtubules in living human tumor cells. Mol. Biol. Cell. American Society for Cell Biology; 1999 April; 10(4):947-59. PMCID: PMC25218
Zhang J, Cunningham J J, Brown J S, Gatenby R A. Integrating evolutionary dynamics into treatment of metastatic castrate-resistant prostate cancer. Nat Commun. Springer US; 2017 Nov. 16; 8(1):1-9. PMCID: PMC5703947

What is claimed is:

1. A method of personalized treatment of prostate cancer recurrence in a subject following primary treatment comprising
(a) Measuring prostate specific antigen (PSA) levels in the patient
(b) Applying the measured PSA levels to a prostate cancer dynamic model;
(c) calculating a patient-specific responder parameters;
wherein intermittent on-an off-androgen deprivation therapy (IADT) is paused when PSA levels are less than patient-specific pre-treatment levels; and wherein IADT is resumed when PSA levels are greater than patient-specific pre-treatment levels.

2. The method of claim 1, wherein intermittent on-an off-androgen deprivation therapy (IADT) is paused when PSA levels are less than 50% of pre-treatment levels.

3. The method of claim 1, wherein IADT is resumed when PSA levels are at least 10% greater than pre-treatment levels.

4. The method of claim 1, wherein the PSA levels are blood PSA levels.

5. The method of claim 1, further comprising measuring androgen-independent prostate cancer stem cell (PcaSC) levels and androgen dependent non-stem prostate cancer cell (PcaC) levels and applying the measured values to step b before proceeding to step c.

6. The method of claim 1, comprising repeating steps a-c every 1, 2, 3, 4, 5, 6, 7, 8, 9, 10, 11, 12, 13, 14, 15, or 16 weeks.

7. The method of claim 1, wherein the primary treatment is surgical resection of the tumor, neoadjuvant or adjuvant androgen-deprivation therapy.

8. The method of claim 1, wherein the method is performed after the patient begins IADT.

9. The method of claim 8, wherein the IADT further comprises the administration of docetaxel or abiraterone acetate (AA).

10. The method of claim 9, further comprising calculating docetaxel or AA cytotoxicity and adjusting dosage and timing of docetaxel or AA.
